(19) United States
(12) Patent Application Publication
(10) Pub. No.: US 2020/0001104 A1
(43) Pub. Date: Jan. 2, 2020

(54) SCALABLE MAGNETOMECHANICAL SCHEMES AND DEVICES FOR REMOTE CONTROL OF MECHANOSENSITIVE CELLS

(71) Applicant: Massachusetts Institute of Technology, Cambridge, MA (US)

(72) Inventors: Polina Olegovna Anikeeva, Somerville, MA (US); Alexander William Senko, Cambridge, MA (US); Danijela Gregurec, Cambridge, MA (US); Pooja D. Reddy, Wellesley, MA (US)

(21) Appl. No.: 16/427,911

(22) Filed: May 31, 2019

Related U.S. Application Data
(60) Provisional application No. 62/679,170, filed on Jun. 1, 2018.

(51) Int. Cl. A61N 2/06; A61K 41/00; C12N 5/079; C12N 5/0793; C12N 13/00

(52) U.S. Cl. CPC A61N 2/06; A61K 41/0052; C12N 13/00; C12N 5/0619; C12N 5/0622

(57) ABSTRACT

The present invention provides novel methods and devices for the remote stimulation of mechanosensitive cells using magnetically-induced forces that are significantly lower in magnitude than currently possible. Novel biocompatible anisotropic magnetic nanodiscs allow for remote magnetomechanical stimulation of cell signaling in mechanosensitive cells at scale without compromising cell viability. The methods and devices of the present invention allow for investigation of mechanoreception in general, and in particular, for studies in neurological, neurodegenerative; and neuromuscular diseases.

### RELATED APPLICATION

This application claims the benefit of U.S. Provisional Application No. 62/679,170, filed on Jun. 1, 2018. The entire teachings of the above application are incorporated herein by reference.

### GOVERNMENT SUPPORT

This invention was made with Government support under Grant No. HR0011-15-C-00155 awarded by the Defense Advanced Research Projects Agency, Grant No. DMR-1419807 awarded by the National Science Foundation, and Grant No. 1-R01-MH111872-01 awarded by the National Institutes of Health. The Government has certain rights in the invention.

### TECHNICAL FIELD OF THE INVENTION

The present invention relates to medicine and cell biology. More particularly, the present invention relates to methods and devices for remote magnetomechanical control of mechanosensitive cells using magnetic nanodiscs at volumes required in experimental and clinical settings.

### BACKGROUND OF THE INVENTION

Minimally invasive control of cell signaling with magnetic fields is being explored in basic studies of the nervous and immune systems¹⁻⁵. Wireless schemes based on hysteretic heating of magnetic nanoparticles in high-frequency alternating magnetic fields (AMFs) have already permitted modulation of neural activity^{2,6} and cancer theranostics in vivo⁷. However, despite these advances, the potential off-target heating effects and challenges in scaling of high-frequency AMF apparatuses^{8,9} impede universal adoption of magnetic hyperthermia in biomedical research.

To date electricity^{10,11,} ultrasound¹², light¹³, electric fields¹⁴, and magnetic fields (MFs)^{2,6} have been applied to modulate cell behavior. Due to the low conductivity and negligible susceptibility of biological matter, MFs allow for control of cell signaling arbitrarily deep within the bodyl^{5,4}. Although transcranial magnetic stimulation with low-frequency, high-amplitude MFs can induce local currents in superficial brain structures³, low-amplitude, high frequency alternating magnetic fields (AMFs) can be coupled to magnetic nanoparticle transducers to locally dissipate heat and excite temperature-sensitive neurons in deep tissues^{2,6}. The power demands of radiofrequency AMF apparatuses, however, pose a challenge to scaling of magnetothermal stimulation to clinically relevant volumes.

Magnetomechanical control of cell activity has been similarly explored, but has primarily relied on magnetic field gradients 100-1000 T/m produced over volumes 10⁻⁹-10⁻⁵ cm^{3 1,16,17}. In larger volumes, MFs have been transduced into torques by permalloy microdiscs and applied to mediate aggregation of isotropic magnetic nanoparticles to trigger apoptosis in cancer cells¹⁸⁻²⁰.

Therefore, a need remains for scalable magnetomechanical schemes and devices that can be used in conjunction with low magnitude magnetic fields over large volumes to control and investigate cell activity and signaling, and which do not compromise cell viability.

### SUMMARY OF THE INVENTION

The inventors have discovered novel methods and devices that provide for the remote magnetomechanical control of mechanosensitive cells. The methods and devices of the invention are used in combination with low-magnitude magnetic fields which are significantly lower in magnitude than currently possible. Specifically, the present invention provides novel methods and biocompatible anisotropic magnetic nanodiscs, referred to herein as “MNDs” for remote magnetomechanical control of mechanosensitive cells at scale without compromising cell viability. The methods and devices of the present invention allow for investigation of inechanoreception in general, and in paiticular, for studies in neurological, neurodegenerative, and neuromuscular diseases. The methods and devices of the invention also allow for novel approaches in the treatment of diseases and disorders.

Various embodiments relate generally to methods and devices involving MNDs. In one embodiment, the invention provides a method for mediating remote control of cell signaling in a biological sample, comprising the steps of:

(i) contacting the biological sample with magnetic nanodiscs; and

(ii) applying a magnetic field to the biological sample,

wherein the applied magnetic field is of low amplitude and low frequency.

In one embodiment, the method provides for remote transduction of magnetomechanical stimuli in a cell membrane of a biological sample. In one embodiment, the cell membrane comprises a mechanosensitive channel. In one embodiment, the method provides for remote modulation of ion influx through mechanosensitive ion channels in the biological sample.

In one embodiment, the magnetic nanodiscs of the method are comprised of magnetite. In one embodiment, the magnetic nanodiscs of the method exhibit colloidal stability. In one embodiment, the magnetic nanodiscs of the method are anisotropic. In one embodiment, the magnetic nanodiscs of the method are biocompatible.

In a further aspect, the method is scalable to large volumes without deleterious effects on the biological sample.

The instant invention also provides a device for mediating remote control of cell signaling in a biological sample in the presence of a weak magnetic field of low amplitude and slow-varying frequency, said device being scalable and providing for remote transduction of magnetomechanical stimuli in a cell membrane of the sample, wherein said membrane comprises a mechanosensitive channel.

In one embodiment, the device is a magnetic nanodisc comprised of magnetite. In one embodiment, the magnetic nanodisc exhibits colloidal stability. In one embodiment, the magnetic nanodisc is anisotropic. In one embodiment, the magnetic nanodisc is biocompatible.

The invention further provides methods of remotely modulating cell signaling in a subject, such as by modulating ion influx through cell membranes in a subject.

In one embodiment, the invention provides a method for remotely modulating cell signaling in a subject, comprising the steps of:

(i) contacting the subject with magnetic nanodiscs; and

(ii) applying a magnetic field to the subject,

wherein the applied magnetic field is of low amplitude and low frequency.

In one embodiment, the method provides for remote transduction of magnetomechanical stimuli in a cell membrane of a subject. In one embodiment, the cell membrane comprises a mechanosensitive channel. In one embodiment, the method provides for remote modulation of ion influx through mechanosensitive ion channels in a subject. In other aspects of the method, the remote, weak, slow-varying magnetic field targets a specific area of the subject.

The invention further provides a method for modulating the permeability of an inorganic membrane or filter. In one embodiment, the permeability of the inorganic membrane can be reversibly or irreversibly modulated by the mechanical motion of embedded or interfaced MNDs.

The methods and devices of the invention provide a means for dissecting at scale the effect of ion signaling on the biochemistry, molecular biology, and physiology of biological samples. In addition, the invention provides for selective modification at scale of cellular activity and function, remotely and non-invasively, both in vitro and in vivo. Such a technique allows one to study the roles of various types of cell signaling in physiological processes, in particular those functions that are, or would be, perturbed by invasive methods, without deleterious effects on the biological sample.

The methods and devices of the invention further provide for selective targeting of cells and tissues in a biological sample or subject in the treatment of a disease or condition. Examples of such methods include, but are not limited to, the mechanical induction of cell death in the treatment of cancer, and the enhanced uptake of exogenously added therapeutics, such as drugs, small molecules, and protein-based therapeutics.

### BRIEF DESCRIPTION OF THE DRAWINGS

FIGS. 1a-1j: FIG. 1a is an illustration depicting the experimental scheme. Slow-varying MF causes magnetic nanodiscs (MNDs) to exert forces on the cell membranes in dorsal root ganglion (DRG) explant cultures, resulting in Ca′ influx as measured by changes in Fluo-4 fluorescence. FIG. 1b-e are scanning electron microscope (SEM) images (b,d, scale bar 1 μm) and selected area electron diffraction (SAED) patterns (c,e, scale bar=100/nm) of hematite (b,c) and magnetite (d,e) nanodiscs. Insets in the SAED images show transmission electron microscope images of the particle on which SAED was performed (scale bar=100 nm). FIG. 1c is a hematite SAED showing single-crystalline hexagonal structure of hematite. FIG. 1e is an SAED showing polycrystalline nature of the magnetite MND. FIG. if illustrates photographs of vials of nanodiscs before and after their conversion from hematite to magnetite and following transfer of magnetite into aqueous media. FIG. 1g-h illustrate vibrating sample magnetometry (g) and powder X-Ray diffraction (XRD) (h) measurements that confirm the complete conversion of the MNDs from hematite to magnetite. FIG. 1i-j illustrate dynamic light scattering of MNDs and isotropic magnetic nanoparticles (IMNPs) of a similar volume and surface chemistry immediately (i) and 5 minutes (j) following suspension in water.

FIGS. 2a-2c shows magnetomechanical stimulation of dorsal root ganglia explants. FIG. 2a is a confocal microscope image of DRG outgrowth transfected with Lenti-CaMKIIα::mCherry (red) and incubated with Fluo-4 (green) that reveals a fraction of cells are excitatory neurons. Scale bar=50 FIG. 2b is an SEM image of DRG explant culture incubated with MNDs. Scale bar=200 μm. Inset: detail of a region of the DRG. Individual MNDs are visible on the surface. Scale bar=2 μm. FIG. 2c is a scale illustration of in-situ magnetomechanical stimulation setup. DRGs loaded with Fluo-4 are exposed to a 5 Hz, 23 mT MF from a large solenoid that surrounds the inverted microscope imaging chamber. Scale bar=4 cm.

FIGS. 3a-i: FIG. 3 illustrates calcium influx in DRG explant cultures in response to slow-varying MF. FIG. 3a illustrates fluorescent Ca′ images collected during magnetomechanical stimulation. Heat map represents fluorescence intensity. Scale bar=100 μm. FIG. 3b illustrates the temperature of the Tyrode's solution as monitored during a magnetomechanical stimulation experiment. Blue bars mark 10 s stimulation epochs. Shaded area represents standard error (s.e.m., n=11 trials). FIG. 3c is a bar graph illustration of fractions of cells responding to MF in the absence and presence of MNDs. The number of cells incubated with MNDs that respond to MF is significantly greater compared to controls as confirmed by one-way ANOVA (n=6, F_{3,20}=91.4, p=7.6×10⁻¹²) and Tukey's honest significant difference (HSD) test (tested at p<0.05 and p<0.001). Error bars represent standard deviation. FIG. 3d-f illustrate Ca²⁺ influx into the cells decorated with MNDs in response to MF. FIG. 3g-i illustrate Ca²⁺ influx into the cells in response to MF without MNDs. FIG. 3d,g illustrate the average ΔF/F₀ trace of all 300 cells per condition. (Arrows in d correspond to the time points shown as images in a). Raw fluorescence traces have been processed to reveal Ca²⁺ transients (FIG. 13 and Methods). Shaded area represents standard error (s.e.m., n=300 cells). FIG. 3e,h are Raster plots indicating the times at which ΔF/F₀ for each cell exceeds the 10a threshold. FIG. 3f,i are histograms showing the number of cells responding within a given 1 s bin.

FIGS. 4a-e illustrates magnetomechanical stimulation in the presence of channel blockers. FIG. 4a: The mechanoreceptor channel blocker Gadolinium(III) ion (Gd³⁺) increases Ca²⁺ influx upon magnetomechanical stimulation at 100 μM concentration, and nearly eliminates Ca²⁺ activity at 1 mM. FIG. 4b: The channel blocker ruthenium red (RuR) increases Ca′ response to magnetomechanical stimulus at 10 μM concentration, while at 100 μM RuR concentration Ca²⁺ response returns to baseline levels. FIG. 4c,d: Sodium channel blocker tetrodotoxin (TTX, c) and gap junction blocker palmitoleic acid (PA, d) do not affect Ca²⁺ response. Shaded areas represent MF pulses. FIG. 4e: A significantly lower fraction of cells responds to magnetomechanical stimulation when incubated with 1 mM Gd³⁺ as confirmed by one-way ANOVA (n=6, F_{2,15}=23.9, p=2.2×10⁻⁵) and Tukey's HSD test (tested at p<0.05 and p<0.001). No significant difference in the number of responders was observed for cells incubated with RuR, TTX, or PA, as confirmed by one-way ANOVA (n=6, F_{2,15}=3.6, p=0.054; n=6, F_{2,15}=0.2, p=0.80; n=6, F_{2,15}=1.4, p=0.28; respectively).

FIGS. 5a-b illustrates Magnetite nanodisc (MND) and isotropic magnetite nanoparticle (IMNP) size distributions. a, Histograms of the MND diameters and thicknesses as measured via TEM. b, Histogram of the IMNP diameters. The average MND diameter and thickness were 218 nm±24 nm (standard deviation) and 41 nm±9.5 nm, respectfully. The average IMNP diameter was 100 nm±15 nm. The average volumes of the MNDs and IMNPs were 1.5×10⁶ nm³±4.3×10⁵ nm³ and 9.9×10⁵ nm³±2.6×10⁵ nm³, respectfully. The IMNPs were 66% of the volume of the MNDs.

FIGS. 6a-b illustrates high resolution transmission electron microscope (TEM) images of MNDs. a, an individual MND, showing grain boundaries. The black box indicates the boundaries of the higher magnification image shown in b. The black boxes inset in b are enlarged subsets of the same image to reveal the crystal orientation of the overlaid grain.

FIGS. 7a-c illustrates Magnetite nanoparticles with a nearly isotropic shape and volume similar to MNDs. a, TEM image of isotropic magnetite nanoparticles (IMNPs). b, Single particle electron diffraction pattern of an IMNP that reflects its cubic crystal structure. Inset: TEM image of the particle used for the diffraction pattern c, vibrating sample magnetometry data for IMNPs (saturation magnetization is ˜76 emu/g).

FIG. 8 illustrates rat dorsal root ganglion (DRG) explant culture one week following seeding. Stained with anti-S100 antibody (a marker of Schwann cells). Scale bar, 500 μm.

FIGS. 9a-c illustrates in-situ magnetomechanical stimulation setup. A custom stage for an Olympus inverted microscope was constructed in order to allow the cells to be both within the bore of the solenoid and within the working distance of the microscope objective. a, custom stage with raised center platform for the DRG. This stage is mounted to the table to mechanically separate it from any vibrations of the solenoid b, the same setup with the addition of a microscope-mounted platform to hold the solenoid c, solenoid in place. 24 cm ruler for scale.

FIG. 10 illustrates magnetic field simulation. Finite element method magnetics (FEMM) simulation of solenoid used for production of slow varying MF. Center region represents the range of possible positions of the DRG sample. The field magnitude across this region is 22.9±0.1 mT (range) and the field gradient magnitude is 0.43±0.01 T/m (range).

FIGS. 11a-f illustrates calcium imaging analysis of magnetomechanical stimulation controls. a-c, Magnetomechanical stimulation experiment control with no MNDs and no AMF. FIG. 11d-f, control with MNDs but no AMF. a,d, Average ΔF/F₀ trace of all 300 cells per condition. Shaded area above and below line represents standard error (s.e.m., n=300 cells). b,e, Raster plots indicating the times at which each cell exceeds the 106 ΔF/F₀ threshold. c,f, Histograms showing the number of cells responding at any given time.

FIGS. 12a-c illustrates distribution of times to first calcium response after AMF application. Histograms and box plots of time to first exceed the 106 ΔF/F₀ threshold after the application of AMF. Each plot represents one of the three AMF pulses applied per trial. a, First pulse, median response time 5.6 s. b, First pulse, median response time 7.1 s. c, First pulse, median response time 6.1 s.

FIGS. 13a-d illustrates two different methods for calculating ΔF/F₀. a-b, F₀ is defined as the very first raw fluorescence value. Blue bars indicate 10 s epochs in which AMF (5 Hz, 23 mT) is being applied. c-d, F₀ is calculated using the algorithm described in the methods section. In this method, instead of a single F₀ value for the whole trace, F₀ is calculated separately for each point. It is the smallest value within a window of time from the point being analyzed. This method also employs an exponentially weighted average filter. The result is that the large calcium transients are preserved while other features are smoothed out. The dotted black lines shown indicate the threshold beyond which the cell is considered to be responding. The threshold is defined as 10 times the standard deviation of the baseline established within the period before the field is first applied. a,c Trace from a cell that has not incubated with MNDs. b,d Trace from a cell that has been incubated with MNDs.

FIGS. 14a-f illustrates calcium imaging analysis of mCherry magnetomechanical stimulation. No significant difference is observed in the response to magnetomechanical stimulation between cells loaded with Fluo-4 (green, a-c), and the subset of the same cells that expressed mCherry after transfection with Lenti-CaMKIIα:mCherry (red, d-f).

FIGS. 15a-jj illustrates calcium imaging analysis of pharmacological influence on magnetomechanical stimulation. Gd′ was applied at doses of 0 μM, 100 μM, and 1000 μM (a-c, d-f, and g-i, respectively). Ruthenium red was applied at doses of 0 μM, 10 μM, and 100 μM (j-l, m-o, and p-r, respectively). Tetrodotoxin was applied at doses of 0 μM, 0.1 μM, and 1 (s-u, v-x, and y-aa, respectively). Palmitoleic acid was applied at doses of 0 μM, 30 μM, and 300 μM (bb-dd, ee-gg, and hh-jj, respectively).

FIGS. 16a-b illustrates cell viability in cells subject to magnetomechanical stimulation with MNDs vs. controls. a, no significant difference in viability after magnetomechanical stimulation was observed, as confirmed by one-way ANOVA (n=6, F_{3,20}=2.3, p=0.10) and Tukey's honest significant difference post-hoc criterion (tested at p<0.05). b, the same test performed 24 hours after magnetomechanical stimulation also showed no significant difference as confirmed by a single-tailed t-test assuming unequal variances (n=6, p=0.34). Scale bars represent standard error.

### DETAILED DESCRIPTION OF THE INVENTION

For convenience, certain terms employed in the entire application (including specification, examples, and appended claims) are defined throughout the specification. Unless defined otherwise, all technical and scientific terms used herein have the same meaning as commonly understood by one of ordinary skill in the art to which this invention belongs.

While the specification concludes with claims that particularly point out and distinctly claim the invention, it is believed the present invention will be better understood from the following description. The present invention may comprise, consist of, or consist essentially of the essential elements and limitations of the invention described herein, as well any of the additional or optional ingredients, components, or limitations described herein.

The components and/or steps, including those, which may optionally be added, of the various embodiments of the present invention, are described in detail below.

All documents cited are, in relevant part, incorporated herein by reference; the citation of any document is not to be construed as an admission that it is prior art with respect to the present invention

Except as otherwise noted, all amounts including quantities; percentages, portions, and proportions, are understood to be modified by the word “about”, and amounts are not intended to indicate significant digits.

Except as otherwise noted, the articles “a”, “an”, and “the” mean “one or more”.

Except as otherwise noted, the article “or” means “or”, “and”, and “and/or”.

As used herein, the term “comprising” means that other steps and other ingredients that do not affect the end result can be added. The term “comprising” encompasses the terms “consisting of” and “consisting essentially of”. The compositions, devices, and methods/processes of the present invention can comprise, consist of, and consist essentially of the essential elements and limitations of the invention described herein, as well as any of the additional or optional ingredients, components, steps, or limitations described herein.

Within the present invention it is to be understood that the combinations, methods, devices, or combined uses according to this invention may envisage the simultaneous, sequential or separate use of the steps, components, or devices. In this context, “combination” or “combined” within the meaning of the present invention may include, without being limited, fixed and non-fixed (e.g. free) forms (including kits) and uses, such as e.g. the simultaneous, sequential or separate use of the steps, components, or devices.

As used herein, the term “subject” refers to any organism. Organisms include, without limitation, a human being, animal, plant, single-celled life form, virus, bacterium, fungus, protist, and moneran.

As used herein, the term “biological sample” refers to any cell, population of cells, organ, organ explant, tissue, or tissue explant of a subject.

As used herein, the terms “treat,” “treated,” “treating,” or “treatment” includes the diminishment or alleviation of at least one symptom associated or caused by the state, disorder or disease being treated.

The present invention provides methods and devices for scalable magnetomechanical remote control of cells in a biological sample or subject based on the use of anisotropic magnetic nanodiscs exposed to a magnetic field of low amplitude and low frequency. The biological sample or subject of interest is contacted with magnetic nanodiscs of the invention, such that excitation of the magnetic nanodiscs by a remote magnetic force of low magnitude results in a physical change or disruption in a mechanosensitive cell membrane or membranes of the biological sample or subject. In some instances, the physical change or disruption results in the modulation of ion influx through a mechanosensitive ion channel.

In one embodiment, the invention provides a method for mediating remote control of cell signaling in a biological sample, comprising the steps of:

(i) contacting the biological sample with magnetic nanodiscs; and

(ii) applying a magnetic field to the biological sample, and wherein the applied magnetic field is of low amplitude and low frequency.

In one embodiment, the method provides for remote transduction of magnetomechanical stimuli in a cell membrane of a biological sample, comprising the steps of:

(i) contacting the biological sample with magnetic nanodiscs; and

(ii) applying a magnetic field to the biological sample, and

wherein the applied magnetic field is of low amplitude and low frequency.

In one embodiment, the cell membrane comprises a mechanosensitive channel or a mechanosensitive ion channel. In one embodiment, the method provides for remote modulation of ion influx through mechanosensitive ion channels in a sample. In one embodiment, the mechanosensitive channel or mechanosensitive ion channel is selected from an anion and cation channel. In one embodiment, the mechanosensitive channel is selected from a calcium, sodium, potassium, or chloride channel.

In one embodiment, the biological sample includes, but is not limited to, a cell, a population of cells, an organ, an organ explant, a tissue, or a tissue explant. In one embodiment, the biological samples include, but are not limited to, dorsal root ganglia explants, neuron and glial cells. Neuron cells may be selected from Purkinje cells, Granule cells, Motor neurons, Tripolar neurons, Pyramidal cells, Chandelier cells, Spindle neurons, and Stellate cells. In one embodiment, the biological samples include, but are not limited to, glial cells. Glial cells may be selected from oligodendrocytes, astrocytes, ependymal cells, Schwann cells, microglia, and satellite cells. In one embodiment, the biological samples include, but are not limited to, muscle cells, or myocytes. Muscle cells may be selected from skeletal, cardiac, and smooth muscle cells.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes without deleterious effects on the biological sample. In one aspect of this embodiment, the volume is greater than 500 cm³.

The invention further provides methods for remotely modulating cell signaling in a subject, such as by modulating ion influx through cell membranes in a subject.

In one embodiment, the invention provides a method for remotely modulating cell signaling in a subject, comprising the steps of:

(i) contacting the subject with magnetic nanodiscs;

(ii) applying a magnetic field to the subject, and

wherein the applied magnetic field is of low amplitude and low frequency.

In one embodiment, the method provides for remote transduction of magnetomechanical stimuli in a cell membrane of the subject. In one embodiment, the cell membrane comprises a mechanosensitive channel or a mechanosensitive ion channel. In one embodiment, the method provides for remote modulation of ion influx through mechanosensitive ion channels in a subject.

In one embodiment, the weak, slow-varying magnetic field is of low amplitude and low frequency. In other aspects of the method, the remote, weak, slow-varying magnetic field targets a specific area of the subject.

In one embodiment, the subject includes, but is not limited to, any organism. In one embodiment, the organisms include, without limitation, a human being, animal, plant, single-celled life form, virus, bacterium, fungus, protist, and moneran.

In one embodiment, the cell membranes within the targeted area of the subject comprise mechanosensitive channels or mechanosensitive ion channels. In one embodiment, the mechanosensitive channels or mechanosensitive ion channels are selected from anion and cation channels. Such anion and cation channels include, but are not limited to, calcium, sodium, potassium, or chloride channels.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes without deleterious effects to the subject. In one aspect of this embodiment, the volume is greater than 500 cm³.

The present invention also comprises a device for mediating cell signaling or inducing cell membrane perturbations in a biological sample or subject in the presence of a weak magnetic field of low amplitude and slow-varying frequency, said device being scalable and providing for remote transduction of magnetomechanical stimuli in a cell membrane of the sample or subject, wherein said membrane comprises a mechanosensitive channel.

In one embodiment, the device is a magnetic nanodisc. In another embodiment, the magnetic nanodisc is biocompatible. In one embodiment, the magnetic nanodiscs are anisotropic. In one embodiment, the magnetic nanodiscs are comprised of magnetite. In one embodiment, the magnetic nanodiscs exhibit colloidal stability. In one embodiment, the magnetic nanodiscs have a diameter of less than 300 nm.

In one embodiment, the magnetic nanodiscs have a concentration of magnetic nanomaterials of between 3 μg/mL and 300 μg/mL. In one embodiment, the concentration of magnetic nanomaterials in the magnetic nanodiscs is less than 100 μg/mL.

In one embodiment, the cell membranes are mechanosensitive. In one embodiment, the cell membranes comprise mechanosensitive channels or mechanosensitive ion channels. In one embodiment, the mechanosensitive channels or mechanosensitive ion channels are selected from anion and cation channels. Such anion and cation channels include, but are not limited to, calcium, sodium, potassium, or chloride channels.

In one embodiment, the biological sample includes, but is not limited to, a cell, a population of cells, an organ, an organ explant, a tissue, or a tissue explant. In one embodiment, the biological samples include, but are not limited to, dorsal root ganglia explants, neurons, and glial cells. Neuron cells may be selected from Purkinje cells, Granule cells, Motor neurons, Tripolar neurons, Pyramidal cells, Chandelier cells, Spindle neurons, and Stellate cells. In one embodiment, the biological samples include, but are not limited to, glial cells. Glial cells may be selected from oligodendrocytes, astrocytes, ependymal cells, Schwann cells, microglia, and satellite cells. In one embodiment, the biological samples include, but are not limited to, muscle cells, or myocytes. Muscle cells may be selected from skeletal, cardiac, and smooth muscle cells.

In one embodiment, the subject includes, but is not limited to, any organism. In one embodiment, the organisms include, without limitation, a human being, animal, plant, single-celled life form, virus, bacterium, fungus, protist, and moneran.

In one embodiment, the subject or sample comprises a cancerous tissue in a human or an animal. The magnetic nanodiscs cause the death of the cancer cells when a low frequency alternating magnetic field is applied.

In one embodiment, the methods and devices of the invention are used to improve the uptake of therapeutics by cells. Examples of therapeutics include, but are not limited to, drugs, small molecules and protein-based molecules. MNDs and therapeutics are delivered to the same tissue comprised of cells, and the mechanical motion of the MNDs causes the cells of the tissue to endocytose or otherwise take up more of the delivered therapeutic.

In one embodiment, the methods and devices of the invention are used to improve the uptake of therapeutics at the blood-brain barrier. In one embodiment, the device is delivered to the brain directly or via intravenous injection. The device permeabilizes the blood-brain barrier via mechanical motion and improves the transmission of drugs, therapeutics, small molecules, proteins, particles, or any other substances suspended in the blood across the blood-brain barrier.

In one embodiment, the methods and devices of the invention are used for mechanical agitation or mixing of two or more substances in a mixture. Mechanical agitation or mixing of two or more substances using the methods and devices of the invention are useful in the formation of solutions, suspensions, colloids, emulsions, and dispersions. For example, the magnetic nanodiscs of the invention may be added to a mixture comprising a solid and a liquid. In the presence of a magnetic field, the magnetic nanodiscs will gently stir the mixture promoting the dissolution of the solid in the liquid. After mixing is complete, the magnetic nanodiscs are optionally removed from the mixture using magnetic separation. In one embodiment, the magnetic nanodiscs of the invention are removed from the mixture using magnetic separation.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

The present invention also provides a method to treat cancer in a biological sample or subject, comprising the steps of:

(i) contacting the cancer with magnetic nanodiscs;

(ii) applying an alternating magnetic field to the cancer, and

wherein the applied magnetic field is of low amplitude and low frequency.

In one embodiment, the method provides for remote transduction of magnetomechanical stimuli in a cell membrane of the cancer. In one embodiment, the method is scalable to large volumes. In one embodiment, the cancer is in a tissue of the subject. In one embodiment, the subject is an animal or a human being.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes. In one aspect of this embodiment, the volume is greater than 500 cm³.

The present invention also provides a method for the enhanced ake of therapeutics by cells in a biological sample or subject, comprising the steps of:

(i) contacting the cells with magnetic nanodiscs;

(ii) applying an alternating magnetic field to the cells, and

wherein the applied magnetic field is of low amplitude and low frequency and the sample or subject has been exposed to a therapeutic.

In one embodiment, the cells of the biological sample or subject comprise a tissue or tissues. In one embodiment, MNDs and therapeutics are delivered to the same tissue or tissues.

In one embodiment, the tissues comprise the blood-brain barrier. In one embodiment, MNDs of the invention are delivered to the brain directly or via intravenous injection. In one embodiment, MNDs permeabilize the blood-brain barrier via their mechanical motion providing for the improved transmission of therapeutics, small molecules, proteins, drugs, particles, or any other substances suspended in the blood across the blood-brain barrier. In one embodiment, the method is scalable. In one embodiment, the method provides for remote transduction of magnetomechanical stimuli in a cell membrane of the biological sample or subject.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes without deleterious effects on the cells of the sample or subject. In one aspect of this embodiment, the volume is greater than 500 cm³.

The present invention also provides a method for mechanical mixing oft two or more substances, comprising the steps of:

(i) combining the substances to form a combination;

(ii) contacting the combination with magnetic nanodiscs;

(iii) applying an alternating magnetic field to the combination; and

(iv) removing the magnetic nanodiscs from the combination by magnetic separation upon completion of mixing,

wherein the applied magnetic field is of low amplitude and low frequency.

In one embodiment, the method provides mechanical agitation or mixing of two or more substances in a mixture. In one embodiment, the method is useful in the formation of solutions, suspensions, colloids, emulsions, and dispersions. In one embodiment, the magnetic nanodiscs are optionally removed from the mixture using magnetic separation. In one embodiment, the magnetic nanodiscs of the invention are removed from the mixture using magnetic separation. In one embodiment, the method promotes the dissolution of a solid in a liquid. In one embodiment, the magnetic nanodiscs promote mixing by gently stirring a solution comprising a solid and a liquid. In one embodiment, the method is scalable.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes without deleterious effects on the substances comprising the combination. In one aspect of this embodiment, the volume is greater than 500 cm³.

The invention further provides a method for modulating the permeability of an inorganic membrane or filter, comprising the steps of:

(i) contacting the membrane or filter with a magnetic nanodisc;

(ii) applying an alternating magnetic field to the membrane or filter; and

(iii) optionally, placing the membrane or filter between one or more biological samples prior to applying the magnetic field,

wherein the applied magnetic field is of low amplitude and low frequency.

In one embodiment, the MNDs are embedded or interfaced in the membrane or filter. In another embodiment, the permeability of the inorganic membrane is reversibly modulated by the mechanical motion of embedded or interfaced MNDs. In one embodiment; the permeability of the inorganic membrane is irreversibly modulated by the mechanical motion of embedded or interfaced MNDs.

In one embodiment, the amplitude of the applied magnetic field is between 5 and 50 millitesla, between 10 and 40 millitesla, or between 20 and 30 millitesla. In one embodiment, the frequency is between 1 and 10 hertz, or between 3 and 7 hertz. In one embodiment, the amplitude of the applied magnetic field is between 5 and 50 millitesla, and the frequency is between 1 and 10 hertz. In one embodiment, the amplitude of the applied magnetic field is between 10 and 40 millitesla, and the frequency is between 1 and 10 hertz. In one embodiment, the amplitude of the applied magnetic field is between 20 and 30 millitesla, and the frequency is between 1 and 10 hertz. In one embodiment, the amplitude of the applied magnetic field is 23 millitesla, and the frequency is 5 hertz.

In one embodiment, the magnetic nanodiscs of the method are biocompatible. In one embodiment, the magnetic nanodiscs of the method are anisotropic. In one embodiment, the magnetic nanodiscs of the method are comprised of magnetite. In one embodiment, the magnetic nanodiscs of the method exhibit colloidal stability. In one embodiment, the magnetic nanodiscs of the method have a diameter of less than 300 nm.

In one embodiment, the magnetic nanodiscs of the method have a concentration of magnetic nanomaterials of between 3 μg/mL and 300 μg/mL. In one embodiment, the magnetic nanodiscs of the method have a concentration of magnetic nanomaterials that is less than 100 μg/mL.

In one embodiment of the method, the method is scalable to large volumes without deleterious effects on the inorganic membrane, filter, or biological sample. In one aspect of this embodiment, the volume is greater than 500 cm³.

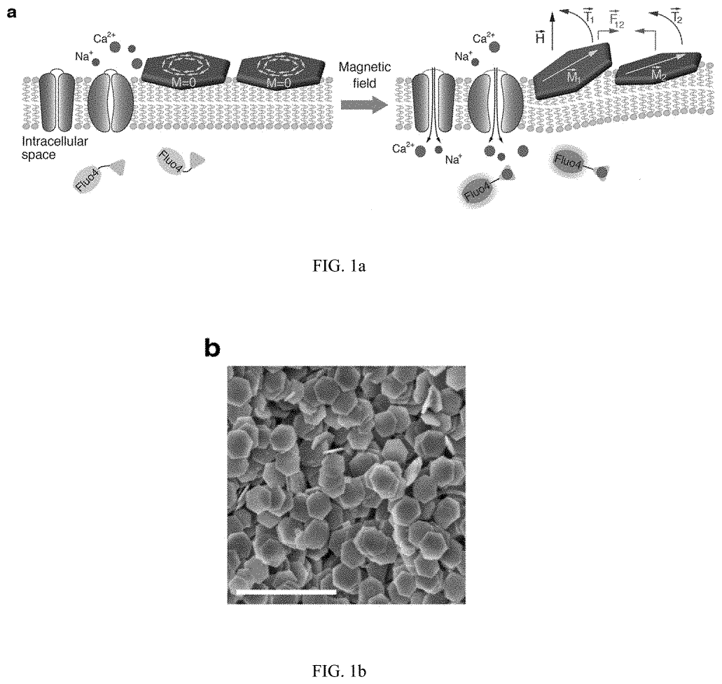

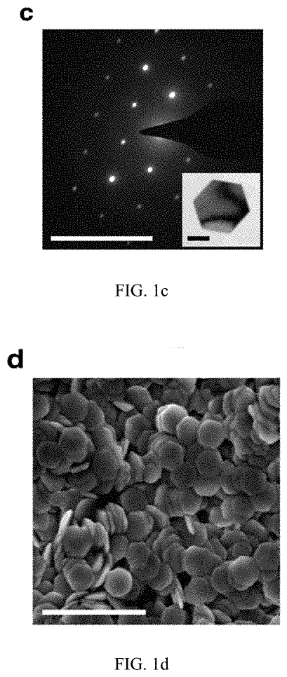

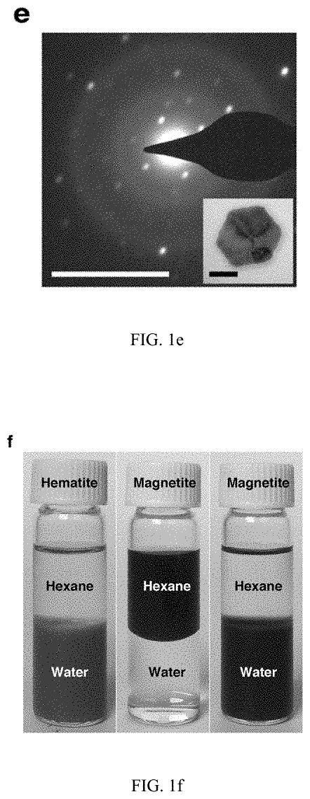

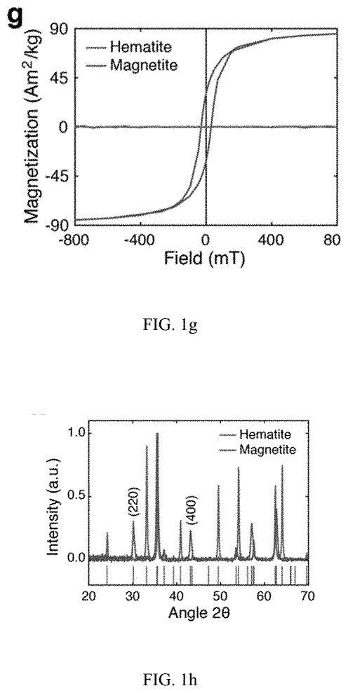

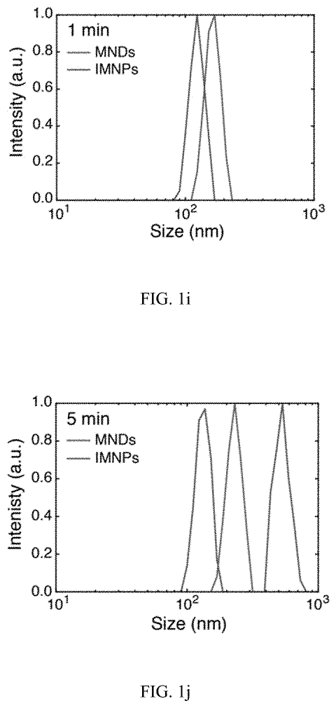

Without being bound by theory, magnetically induced forces of lower magnitude may enable stimulation of mechanosensitive cells without compromising cell viability. The magnetomechanical scheme of the invention relies on colloidally stable magnetite MNDs to mediate ion influx into cells, such as Ca²⁺ influx in neurons and glia, upon application of weak (23 mT), slow-varying (5 Hz) MFs (FIG. 1a).

Anisotropic magnetite MNDs were synthesized at scale and exhibited colloidal stability and biocompatibility in vitro. When interfaced with the cell membranes, MNDs acted as transducers of low frequency (5 Hz) low amplitude (23 mT) magnetic field into mechanical force. This scheme was applied to control calcium influx in genetically intact dorsal root ganglia, which contain both neurons and glia. In contrast to magnetothermal approaches, the system used in this work offers straightforward scalability to large volumes (>500 cm³), requires significantly lower concentrations of magnetic nanomaterials (<100 μg/mL), and is capable of modulating cell calcium without reliance on transgenes.

While metallic iron alloy particles boast high saturation magnetization desirable for magnetomechanical transducers, such materials are likely to oxidize and lose their utility in biological environments. In contrast, magnetite (Fe₃O₄, Mₛ=85 emu/g) nanoparticles are chemically stable in physiological conditions and are commonly used in biomedical applications^{21,15}. The force thresholds for activation of mechanosensitive channels (MSCs) or membrane receptors typically range between 0.2-1 pN²²⁻²⁴. Static and slow-varying MFs can be transduced by magnetic nanoparticles into membrane tension via: (1) torque; (2) inter-particle attraction force; and (3) gradient force⁴. Given the modest gradients in large-volume MF coils needed for experiments in animal models, the latter mechanism is unlikely. MNDs with diameters of hundreds of nanometers exhibit magnetic moments sufficient to produce the torques or inter-particle attraction forces necessary to stimulate MSCs in the presence of low-magnitude MF s (Methods). While isotropic particles of similar volume exhibit poor colloidal stability due to magnetic dipole-dipole interactions²⁵, MNDs can support a magnetic vortex state with zero net magnetization in the absence of MF²⁶.

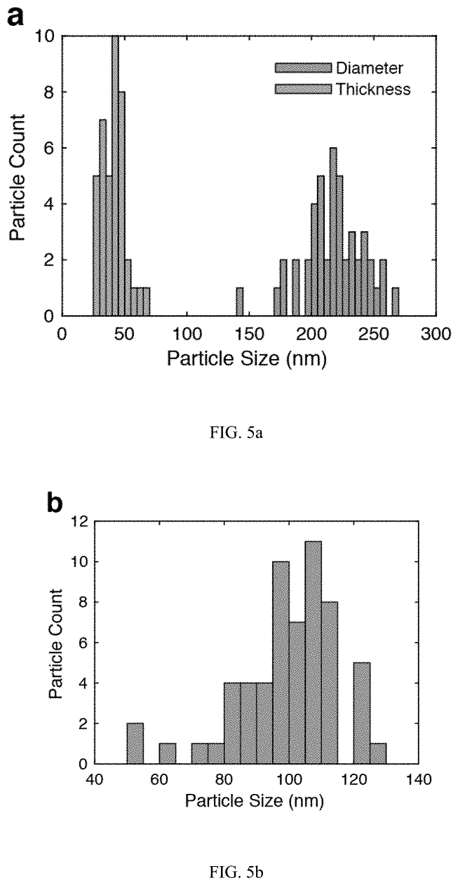

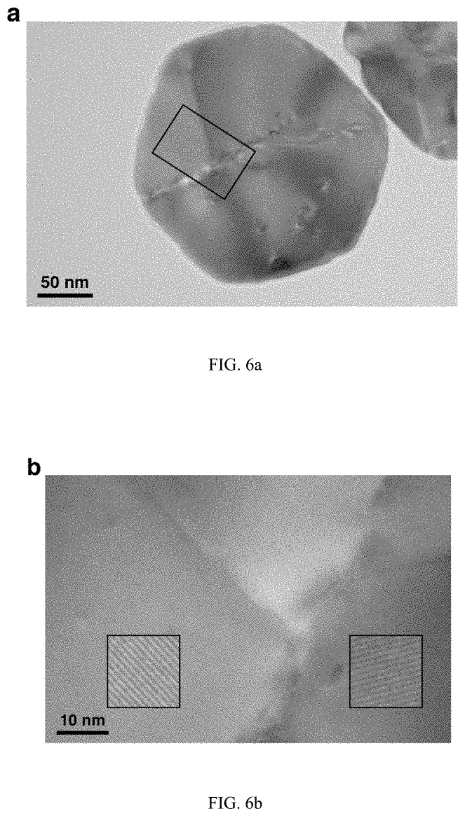

Colloidal synthesis of anisotropic magnetite particles presents a challenge because the cubic symmetry of the inverse spinel lattice implies isotropic particle growth. A two-step synthesis was adapted to first produce hexagonal hematite nanodiscs (α-Fe₂O₃, a canted antiferromagnet, FIG. 1b,c), and then reduced them into magnetite MNDs (FIG. 1d,e and FIG. 5, diameter 218±24 nm and thickness 41±9.5 nm)²⁶. The conversion of hematite to magnetite is manifested in a concurrent change of the solution color, a dramatic increase in magnetization, and the emergence of (220) and (400) peaks in X-ray diffraction spectra (FIG. 1f-h and FIG. 6).

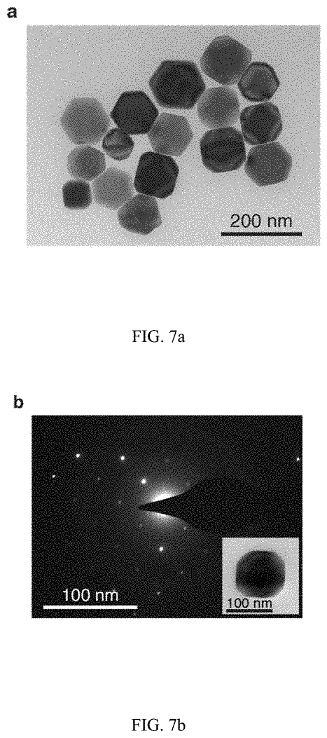

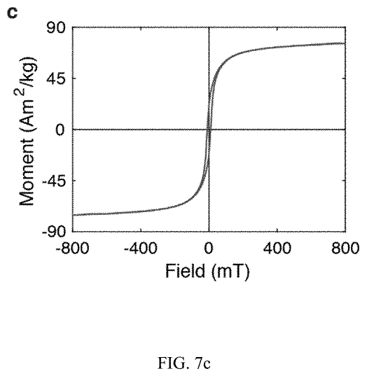

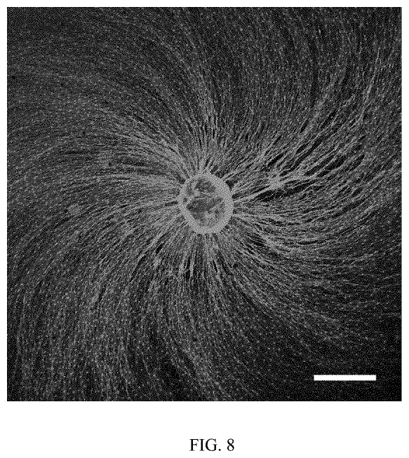

Hydrophobic MNDs coated with oleic acid during the reduction reaction were rendered soluble in aqueous media via surface passivation with a biocompatible amphiphilic ligand poly(maleic anhydride-alt-1-octadecene) (PMAO)²⁷. (Dynamic light scattering corroborated colloidal stability of PMAO-coated MNDs (ζ-potential of −35 mV) and revealed hydrodynamic radii of 167±25 nm (likely an angle-weighted average of the diameter and thickness of the MNDs, FIG. 1i). In contrast to MNDs, isotropic magnetite nanoparticles of similar volume (FIG. 7 2.6×10⁵ nm³, ˜66% MND volume) and ζ-potential (−40 mV) precipitated from solution within minutes (FIG. 1j). Colloidal stability of MNDs can likely be attributed to the reduced inter-particle attraction afforded by the vortex state.

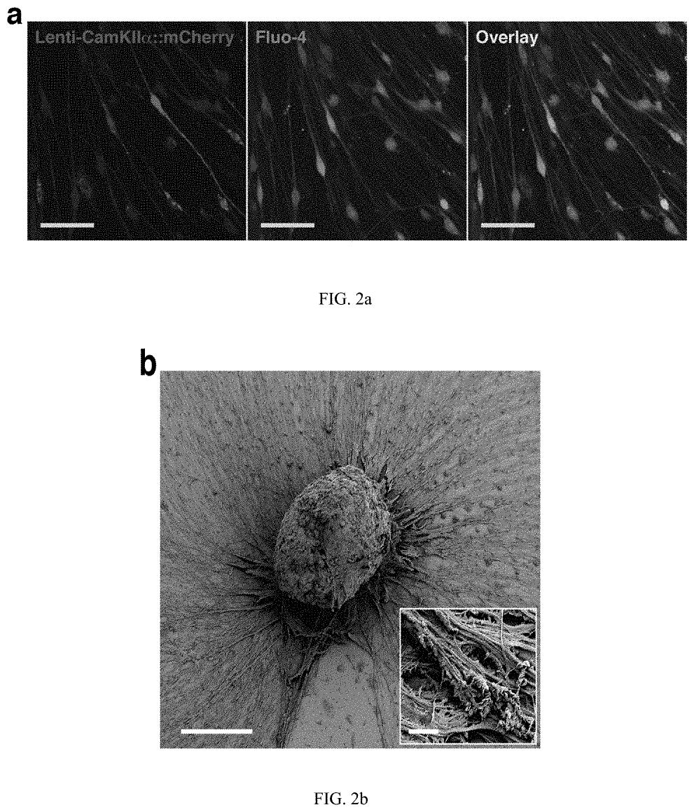

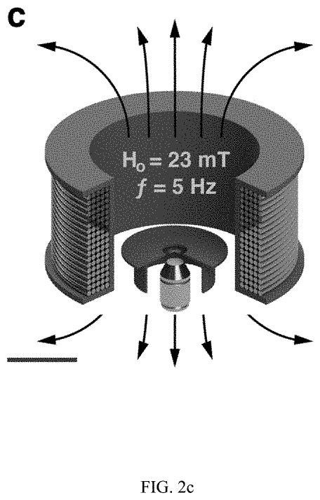

To evaluate the ability of MNDs to act as transducers of magnetomechanical stimuli, we applied this approach to control intracellular Ca²⁺ in one-week old dorsal root ganglion (DRG, FIG. 8) explant cultures containing both neurons and glial Schwann cells, which we hypothesized would represent more physiological models of the neural tissue then the dissociated cultures of neurons or glia. Calcium signaling in both neurons and glia is well-documented^{28,29}, and a minimally invasive, wireless approach for controlling Ca²⁺ influx may not only deliver a neuromodulation scheme but also enable basic studies of glial function in behaving animals. Transfection with a lentiviral vector carrying a fluorescent protein mCherry under a promoter calmodulin kinase II a-subunit (Lenti-CaMKIIα::mCherry) which, in the mammalian brain, is specific to excitatory glutamatergic neurons³⁰, revealed that the DRG outgrowth was composed of neurons positive for CaMKIIα (49±13% of overall population, mean±s.d., n=6 samples), Schwann cells, and possibly neurons not expressing CaMKIIα, (FIG. 2a).

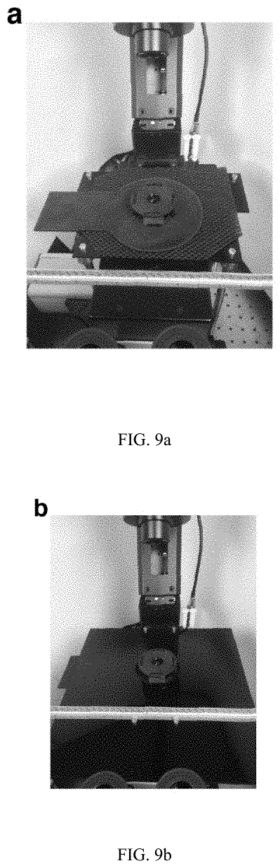

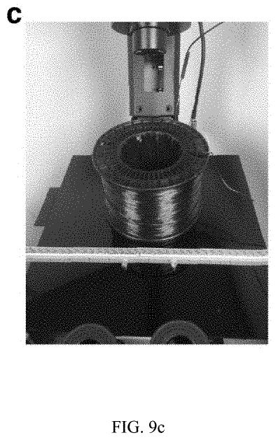

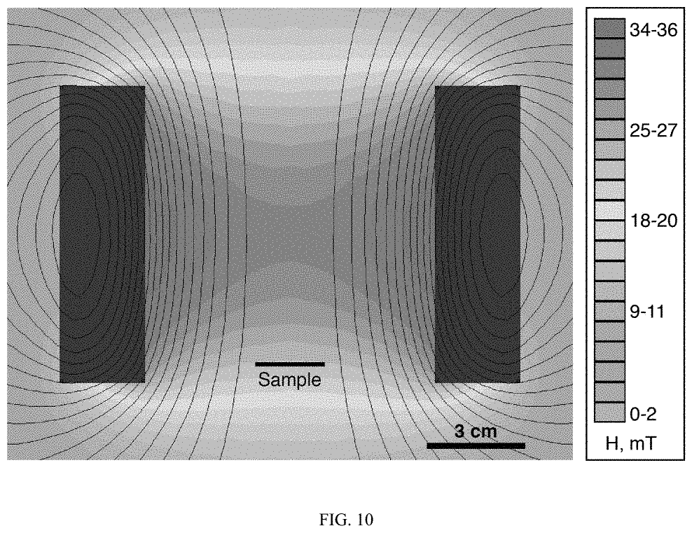

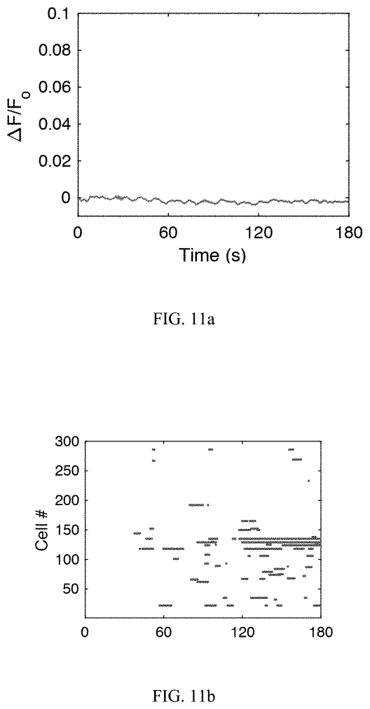

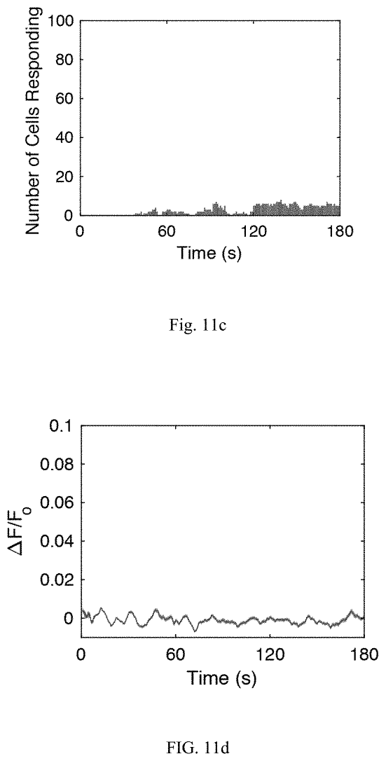

Calcium imaging with fluorescent indicator Fluo-4 was used to quantify the response of the mixed DRG cultures to the magnetomechanical stimulation. Each DRG was incubated with MNDs (60 μg/ml) in Tyrode's solution (FIG. 2b) and imaged while a slow-varying 5 Hz, low-amplitude 23 mT MF was applied in 10 s pulses via a homemade solenoid (FIG. 2c, FIGS. 9 and 10).

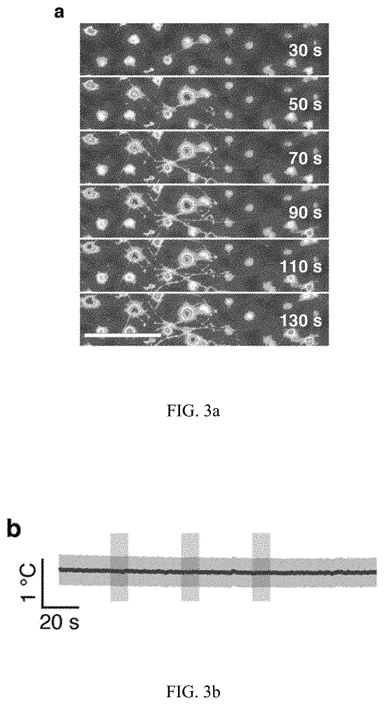

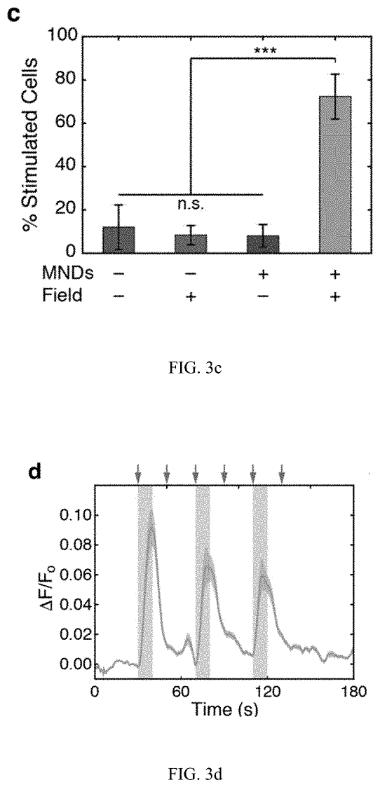

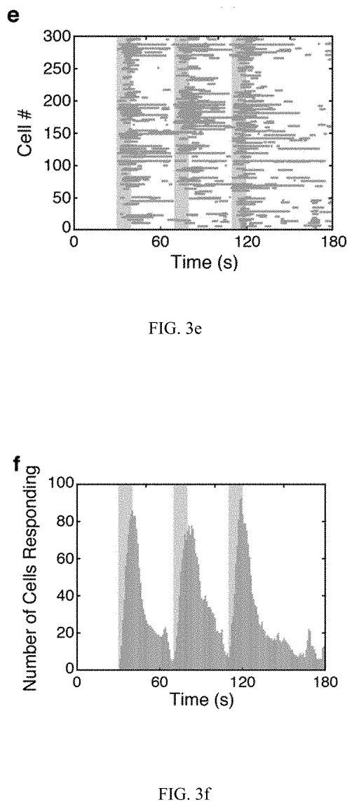

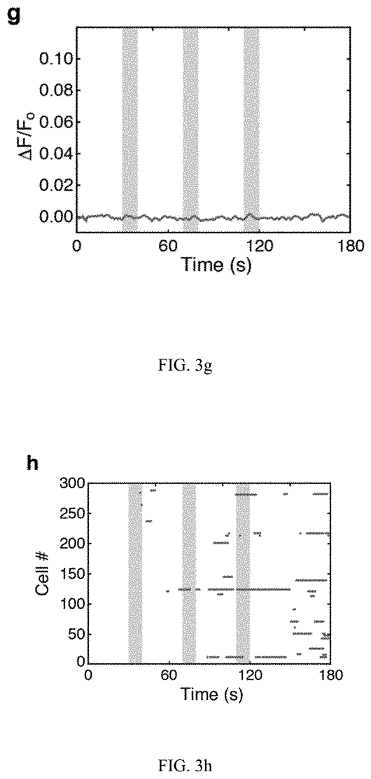

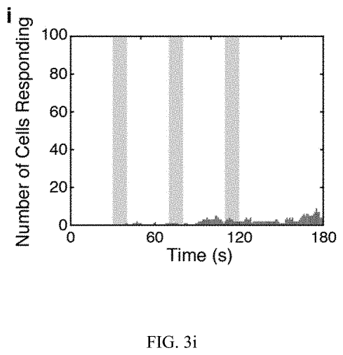

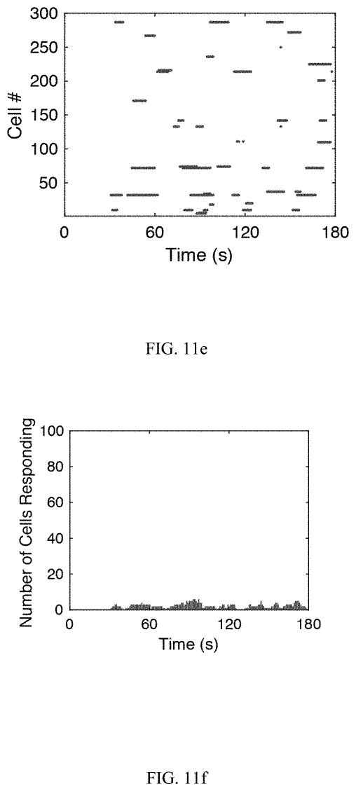

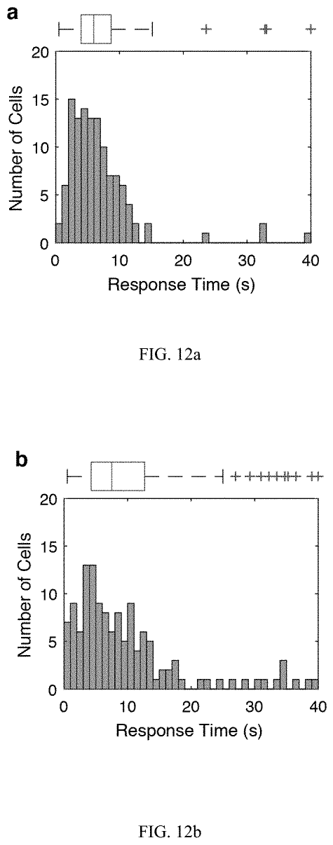

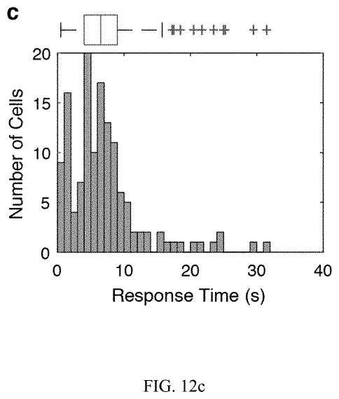

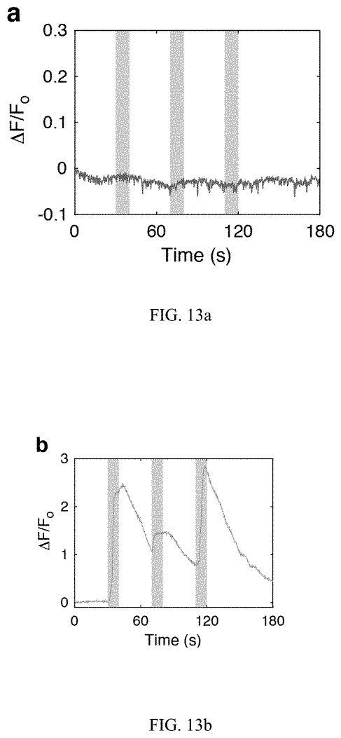

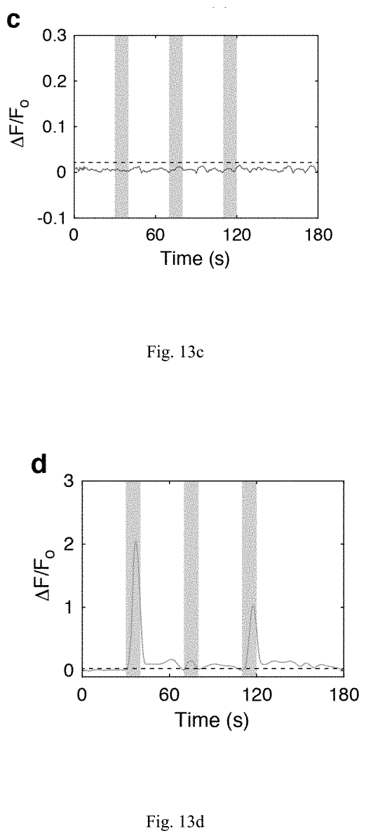

In cultures incubated with MNDs, MF repeatedly evoked Ca′ influx as indicated by a significant increase in Fluo-4 fluorescence intensity normalized to the baseline levels recorded prior to stimulation (ΔF/F₀, FIG. 3a-f). In contrast, negligible response was found in the absence of MNDs (FIG. 3g-i). Note, that temperature remained stable throughout the experiments independent of applied MF (FIG. 3b). Similarly, incubation with MNDs without applied MF did not yield significant changes in intracellular Ca²⁺ (FIG. 11). The latency of Ca²⁺ influx in DRG cells incubated with MNDs was 8±7 s (FIG. 12). To identify cells responsive to MF, standard deviation (a) of baseline fluorescence was calculated for each cell, and then a threshold of 10σ above the baseline was applied to the ΔF/F₀ signal (FIG. 13). In DRGs incubated with MNDs 72±10% (mean±s.d.) of cells responded to MF, while in control samples (without MNDs, MF or either) the population of responsive cells was significantly lower (8±4%, 8±5%, 12±10%, respectively).

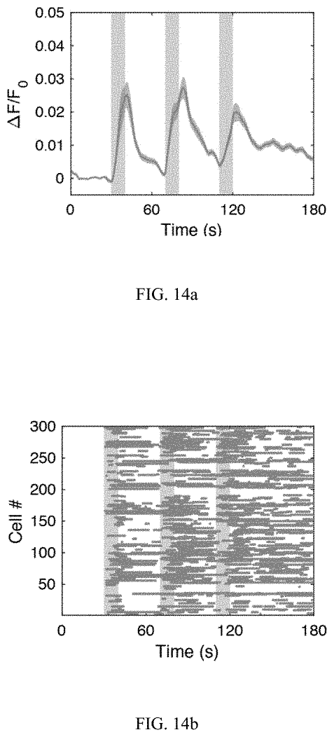

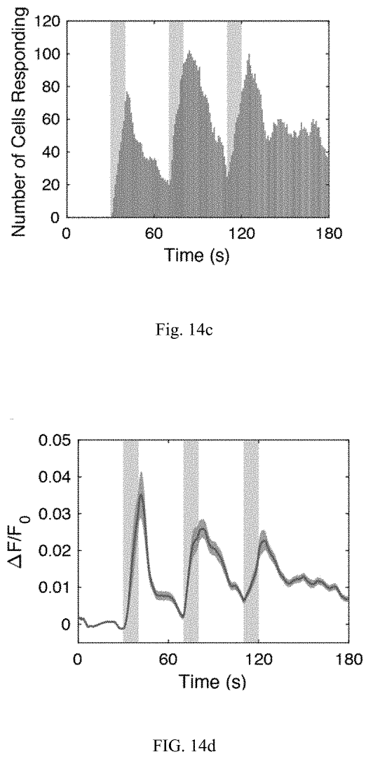

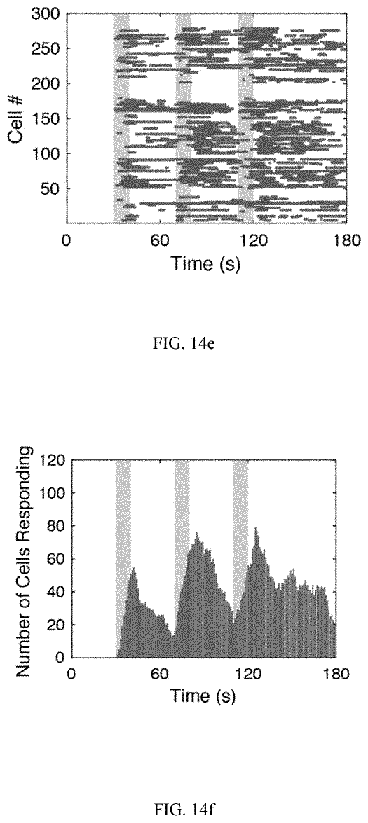

To investigate the effects of magnetomechanical stimulation on neurons and glia, we applied MF to MND-decorated DRGs transduced with Lenti-CaMKIIα::mCherry. No significant differences were identified between the fraction of responsive cells expressing mCherry and the fraction of all live (mCherry⁺ and Fluo-4⁺: 73±14%; all Fluo-4⁺: 76±18% mean±s.d., Student's one-tailed t-test assuming unequal variance, p=0.40), which indicates that neurons and glia are responsive to magnetomechanical modulation in the presence of MNDs (FIG. 14).

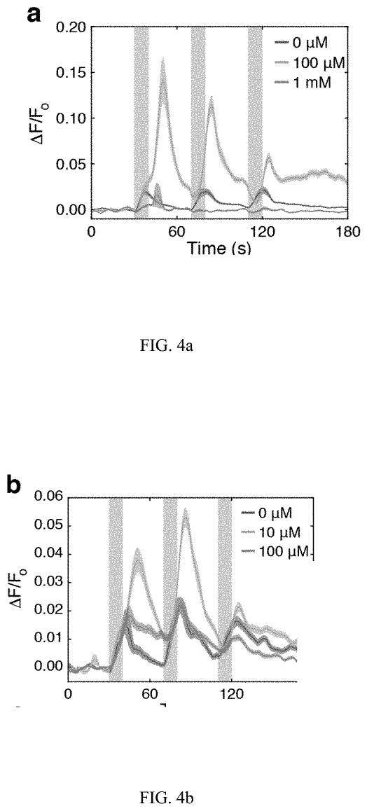

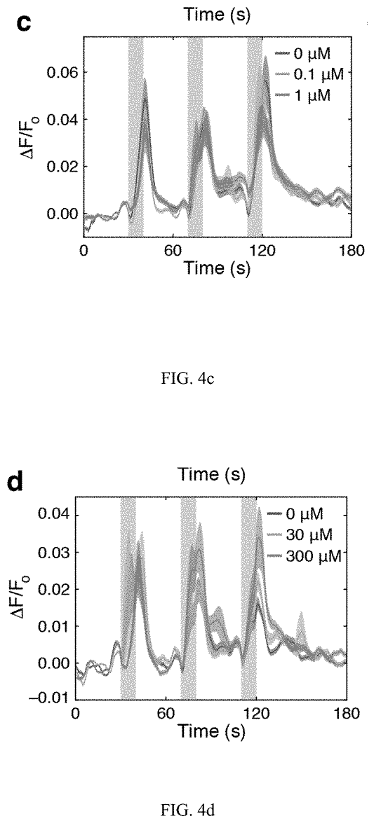

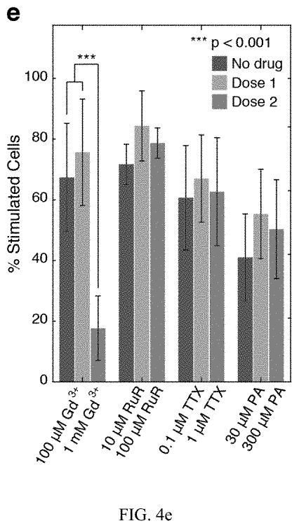

To further explore biophysical mechanisms underlying the observed response to MF in the presence of MNDs, a palette of antagonists was used to block potential mechanoreceptors (FIG. 4). Gadolinium(III) (Gd³⁺) chloride, a nonspecific MSC blocker³¹, produced an enhanced response at low doses (100 μM) and a reduced response at higher doses (1000 μM) (FIG. 4a,e). This biphasic effect of Gd³⁺ has been observed in MSCs from the transient receptor potential (TRP) family³², and could indicate participation of these channels in the observed response. Surprisingly, incubation with ruthenium red (RuR) at 10 μM, an antagonist of TRP channels³³ as well as of the known DRG mechanoreceptor Piezo2³⁴, produced larger ΔF/F₀ changes as compared to experiments without the drug. However, at 100 response magnitude returned to baseline level (FIG. 4b). Although RuR was anticipated to decrease calcium activity, these concentrations of the drug have been reported to potentiate firing in neurons expressing TRP channels³³. Dose-dependent effects of the common mechanoreceptor antagonists RuR and Gd³⁺ indicate that the observed response of DRG cultures to the MF in the presence of MNDs is likely mediated by synergistic action of several mechanoreceptors with varied kinetics, highlighting the potential of this method for mechanobiology studies.

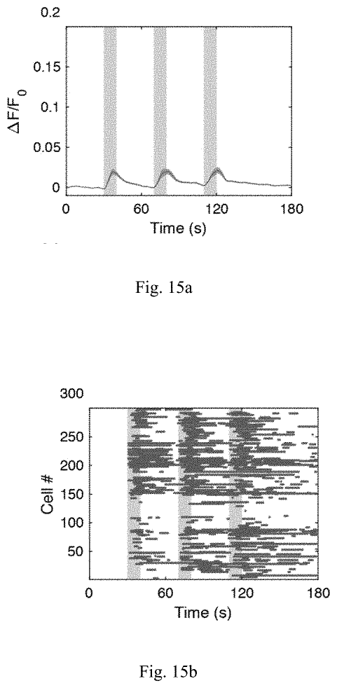

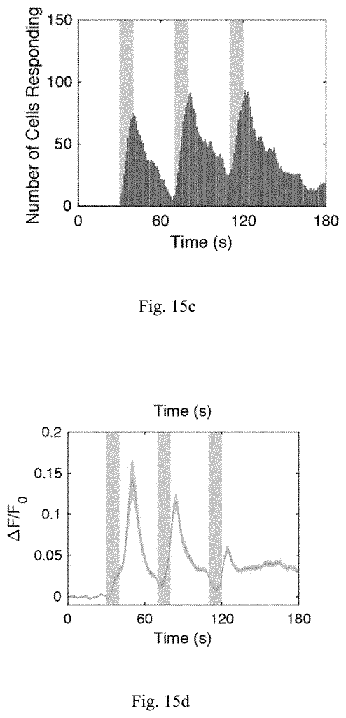

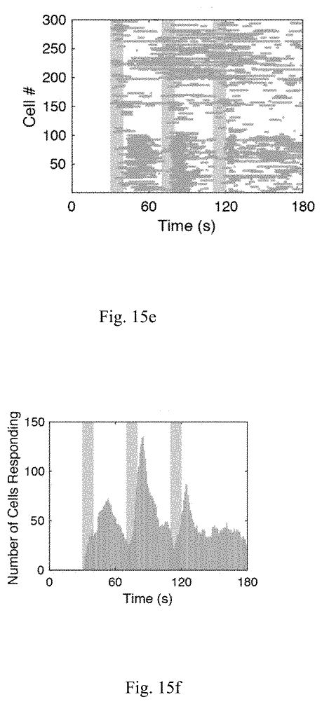

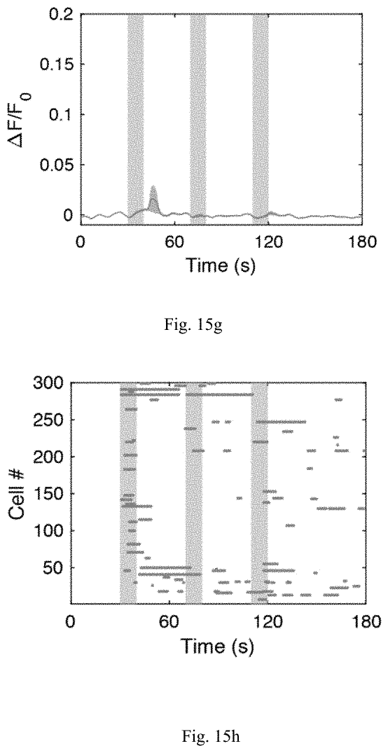

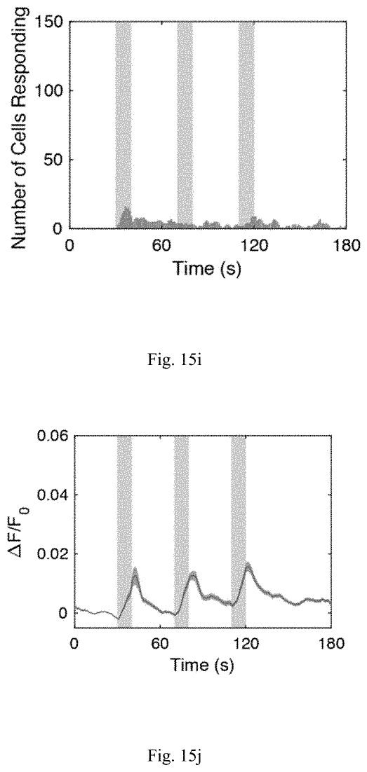

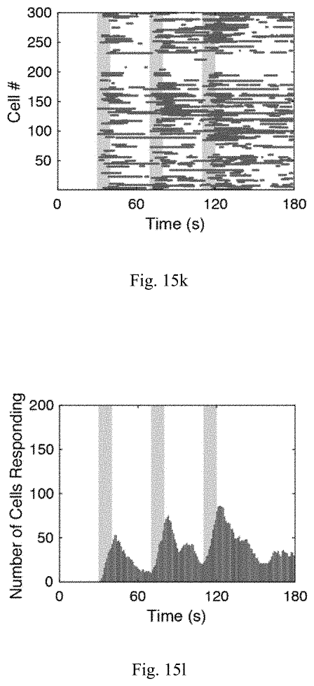

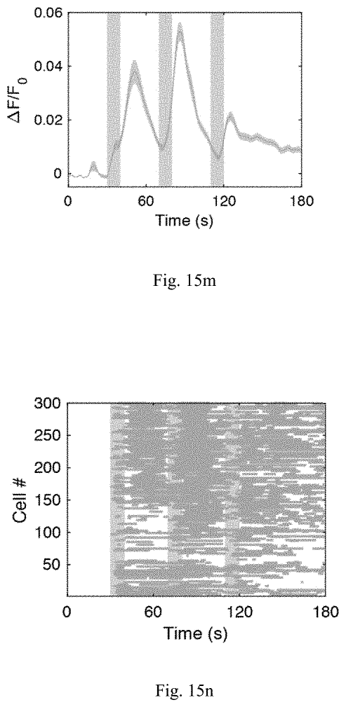

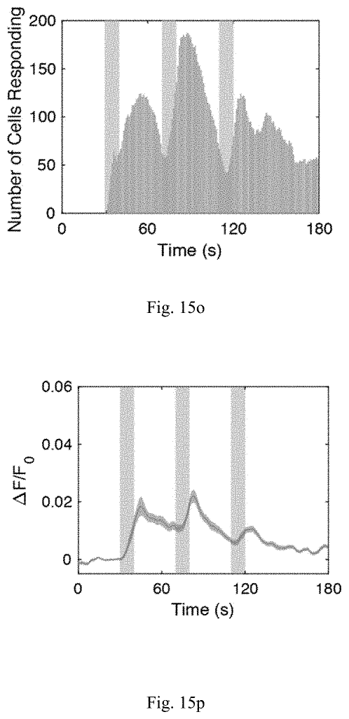

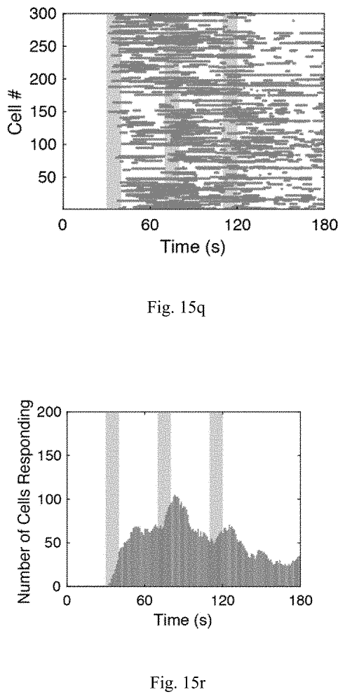

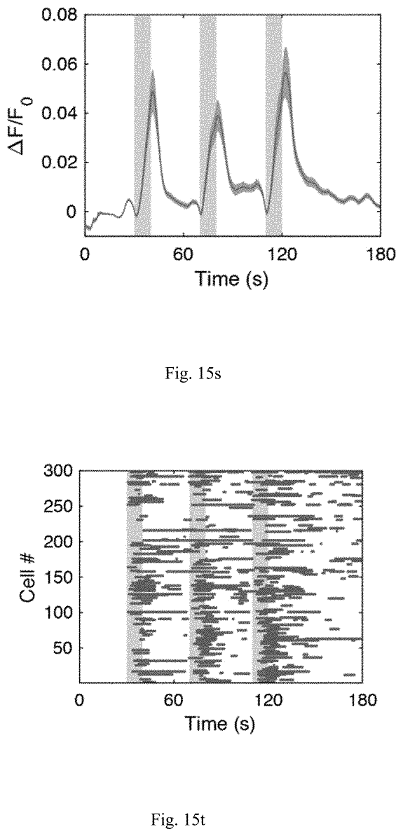

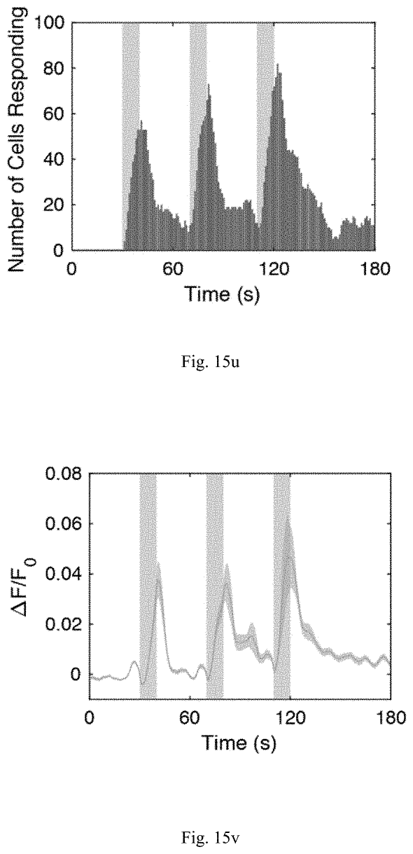

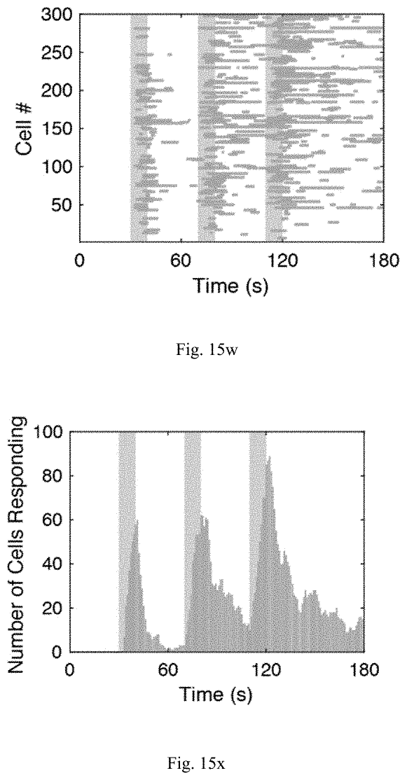

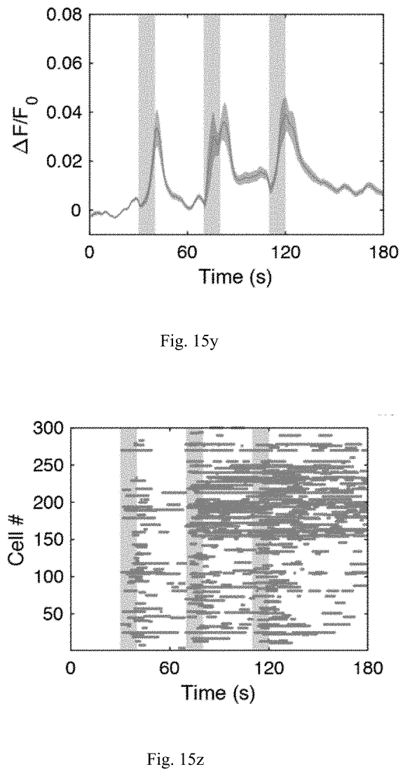

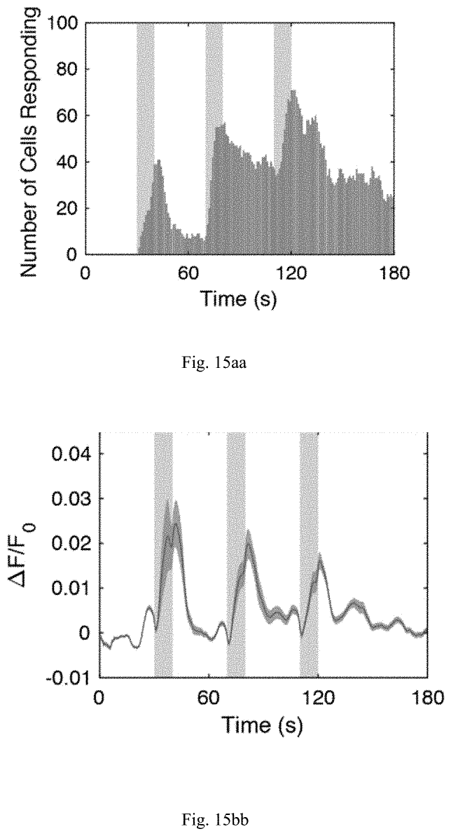

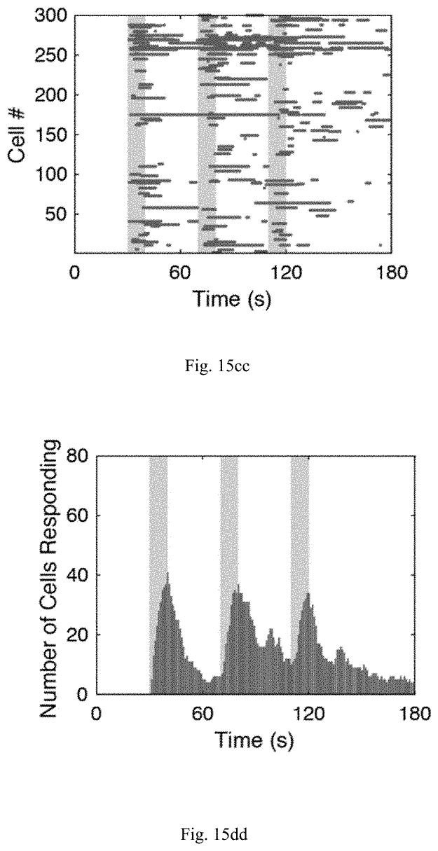

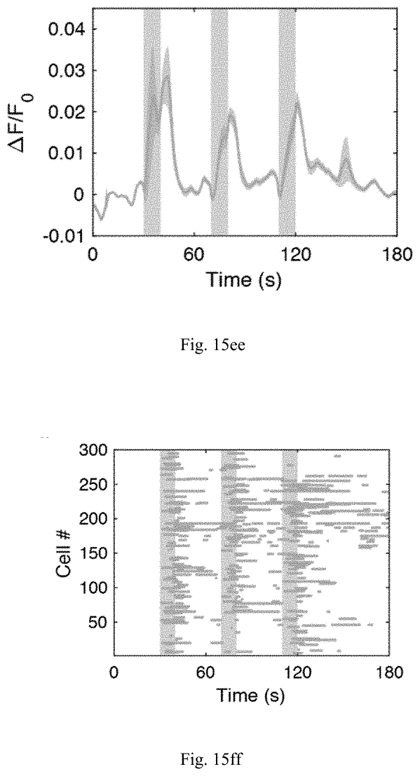

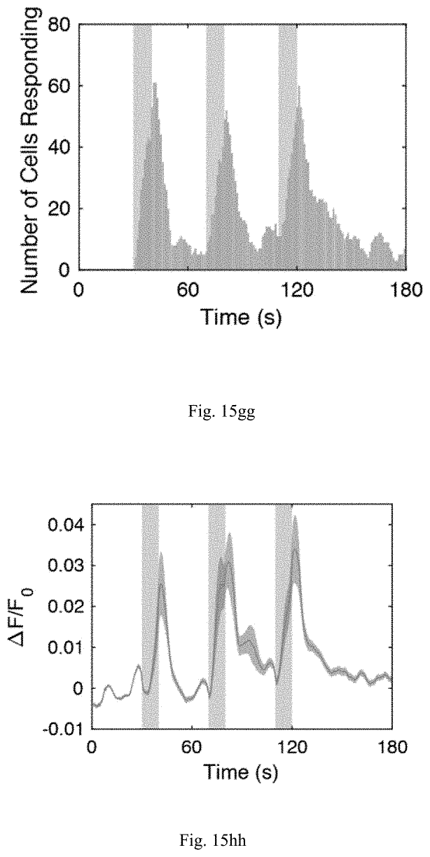

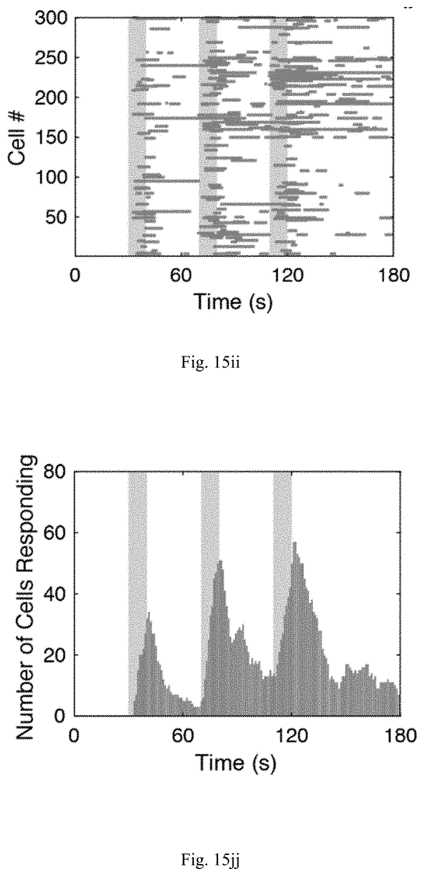

To assess the contribution of electrically mediated network responses in our mature DRG cultures, a sodium channel blocker tetrodotoxin (TTX, 0.1-1 μM) was employed to inhibit action potential firing in neurons, while a gap junction blocker palmitoleic acid (PA, 30-300 μM) was used to prevent glial Ca²⁺ signaling³⁵. Both TTX and PA failed to block the Ca′ influx in cells induced by MF in the presence of MNDs (FIG. 4c-e), indicating that action potentials and glial Ca²⁺ waves do no contribute significantly to the observed response (FIG. 15).

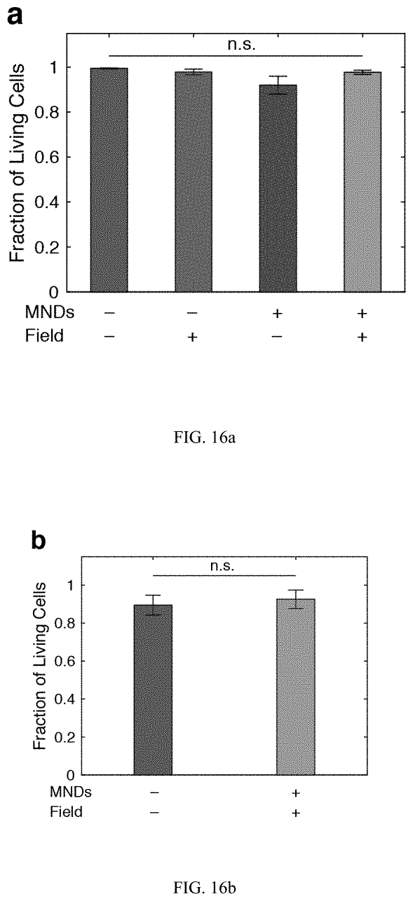

Biocompatibility of magnetomechanical stimulation was evaluated via a membrane permeability ethidium homodimer-1 and calcein-AM assay in DRG cultures immediately after and 24 hours following exposure to MF. No significant differences in membrane permeability were found between the MND-decorated DRGs subjected to MFs and control samples, indicating that the magnetomechanical stimulation does not yield membrane damage (FIG. 16).

In this work, biocompatible anisotropic MNDs were synthesized at scale and exhibited colloidal stability superior to isotropic particles of similar volume likely due to their vortex magnetization. The large induced magnetic moments of the MNDs allowed for magnetomechanical stimulation of neurons and glia in DRG explant cultures over experimental volumes 7 orders of magnitude greater than has been previously achieved with MFs. In addition to providing a tool for investigation of mechanoreception, wireless magnetomechanical stimulation may also allow for regulation of activity of other mechanosensitive cells such as myocytes. Furthermore, the inexpensive, off-the-shelf components used in this study may facilitate broader application of this method in studies of neurological, neurodegenerative, and neuromuscular diseases.

### EXAMPLES

Methods

Magnetic nanodisc synthesis.

Magnetite nanodiscs were synthesized based on a two-step process pioneered by Yang et al.²⁶ in which hematite nanodiscs are first synthesized and then converted to magnetite. To synthesize the hematite disks, 10 mL of absolute ethanol, 0.8 g of sodium acetate, and 0.6 mL of DI H₂0 were added to a glass vial and magnetically stirred until homogenized. Then 0.273 g FeCl₃.6H₂O was added and stirred. The homogenized mixture was moved to a Teflon-lined steel vessel, sealed, and heated to 180° C. for 18 h. Once the reactor cooled to room temperature, the red hematite disk product was washed several times with DI H₂O. The clean particles were then dispersed in ethanol and dried in a vacuum desiccator. To reduce the hematite disks to magnetite, 100 mg of hematite, 20 mL trioctylamine, and 1 g oleic acid were sonicated until homogenized. The mixture was placed in a three-neck flask connected to a Schlenk line and heated at 20° C./min to reflux at 370° C. for 25 min in an atmosphere of H₂ (5%) and Ar (95%). The mixture will turn a metallic silver-black. Once cooled, the mixture was washed several times with hexane. The clean magnetite particles were then dispersed in chloroform and stored in a glass vial.

Isotropic Magnetite Nanoparticle Synthesis.

Large quasi-spherical magnetite particles were synthesized using a modification of a common chemistry³⁶. 1.41 g iron(III) acetylacetonate, 2.26 g oleic acid, and 20 mL benzyl ether were mixed in a flask. The mixture was then evacuated of dissolved gas by applying vacuum in a Schlenk line and then heated at 20° C./min to reflux at 300° C. for 55 minutes in a nitrogen atmosphere. Once the mixture cooled, it was washed several times with equal parts hexane and ethanol. The cleaned particles were then resuspended in hexane and stored in glass vials.

Magnetic nanoparticle phase transfer.

Both magnetite particle syntheses described above produce particles coated with oleic acid, a fatty acid whose exposed hydrocarbon chains make the particles hydrophobic. In order to make them soluble in water, they were coated with poly(maleic anhydride-alt-1-octadecene) (PMAO) (average Mn 30,000-50,000, Aldrich). First, a 10 mg/mL solution of PMAO in chloroform is prepared. Then, this solution is added to dried nanoparticles such that nanoparticle concentration is 1 mg/mL (iron basis). This mixture is then sonicated for one minute and allowed to dry in a vacuum desiccator. When completely dry, TBE buffer was added and the nanoparticles were resuspended via sonication at 80° C. for 2 hours. Then the particles were washed with DI H₂O and stored in DI H₂O in a glass vial.

Dorsal Root Ganglion (DRG) Culture.

12 mm round glass cover slips were coated with 70 uL Matrigel® solution (Matrigel diluted 30× in Neurobasa™-A medium with serum-free B27™ Supplement) overnight prior to DRG seeding. Whole DRGs from PO Sprague-Dawley rat pups were seeded individually on these cover slips in a 24 well plate with 1 mL Neurobasa™-A medium with serum-free B27™ Supplement per well.

Magnetic Field System.

A 10 lb spool of 16 AWG magnet wire was purchased from MWS Wire Industries. The center of this spool was removed to create a large-bore solenoid. A commercial magnetic field (MF) probe was used to measure the field/current relationship for the solenoid, and afterward the current measured via voltage across a 2 ohm shunt resistor connected in series with the solenoid was used to infer the MF amplitude in experiments. The solenoid/resistor circuit was driven by a Crown DC-300 power amplifier with a 3 V p-p 5 Hz input signal from a function generator.

Magnetomechanical Stimulation.

Magnetomechanical stimulation was assessed by performing calcium imaging on DRG explant culture incubated with magnetic nanodiscs and applying low frequency MF in situ. 1 mM Fluo-4 AM (Invitrogen) in DMSO was diluted 1000× in Neurobasa™-A medium with serum-free B27™ Supplement. DRGs were incubated in this solution for 45 minutes and then transferred to Tyrode's solution. 30 uL of magnetite nanoparticles at a concentration of 2 mg/mL were added to each well and the 24-well plate was gently shaken by hand to disperse the particles as they were added. After 15 minutes' incubation time, DRGs were added one by one to a custom imaging chamber surrounded by a solenoid (see FIG. 9) on an inverted microscope and after a 4 min equilibration time, calcium imaging began. During the experiment, slow-varying MF (5 Hz, 23 mT) was applied in three 10 s pulses.

Calcium Imaging Analysis.

Videos of calcium activity were collected in the .vsi format using the Olympus cellSens software. The frame rate was 250 ms and each experiment was 3 min long. The .vsi files were opened in ImageJ with the Bioformats package, and the Threshold,

Watershed, and Analyze Particles functions were used to delineate, separate, and circle cells automatically. The average grayscale value in the ROIs generated this way was exported in a .csv file and processed with MATLAB. A custom MATLAB script was used to convert the average grayscale value of each ROI over time into ΔF/F₀ traces using the algorithm described in Box 1 of Jia et al.³⁷, with τ₀=0.75 s, τ₁=2.5 s, and τ₂=7.5 s, but with the modification that a central moving average and F₀ calculation was used instead of a simple moving average. A second MATLAB script was used for choosing at random subset of 50 cells from each experiment and classifying them as responsive or non-responsive based on whether their ΔF/F₀ trace ever exceeds 10 times the standard deviation of the signal during the period before field is ever applied.

mCherry Transfection and Calcium Imaging Analysis.

Lenti-CaMKIIα::mCherry was packed in-house. After 1 week of growth, 1 μL per DRG was added to the medium. 1 week later, the cells were incubated with Fluo-4 and MNDs and imaged in an inverted microscope while a 5 Hz, 23 mT field was applied. mCherry was imaged in the same region where calcium imaging was performed, such that ROI for both channels could be overlaid. ROIs generated using the mCherry channel were compared with ROIs generated using the Fluo-4 channel, and mCherry ROIs that did not correspond to Fluo-4 ROIs (such as ROIs from neuronal processes expressing mCherry but with relatively little Fluo-4 (compared to somata)) were eliminated. When mCherry ROIs were strictly a subset of Fluo-4 ROIs, calcium imaging analysis and quantification of the relative fraction of mCherry⁺ cells were performed. There were generally fewer than 50 mCherry⁺ cells per field of view, so instead of randomly choosing a subset of 50 cells, all cells were analyzed.

Cell Viability Assay.

The Invitrogen LIVE/DEAD® Viability/Cytotoxicity kit was used to assess whether magnetomechanical stimulation or exposure to MNDs caused cytotoxicity. The kit uses calcein AM (green) to mark live cells and ethidium homodimer-1 (red) to mark dead cells. The stock solutions both diluted in a single mixture containing 400 nM of both dyes. Immediately before, during, and for 45 minutes after stimulation, DRGs were incubated in this solution. Then they were imaged with an inverted microscope, and a MATLAB script was used to process the images: each region imaged was imaged with both red and green filters; the relative abundance of green cells and red cells (by area) was tabulated for each region for each condition, with 100% live cells corresponding to all cells being observed with a green filter and none observed with the red filter.

### Magnetic Forces Calculations

In the presence of static or slow-varying magnetic fields magnetic nanodiscs (MNDs) can transduce tension to cell membranes according to three mechanisms: (1) torque; (2) inter-particle attractive forces; and (3) gradient force. Below we use simple analytical models to calculate relative contributions from these three mechanisms and identify the most likely underlying principle for magnetomechanical control of cell calcium.

Moment of an Individual MND:

The average volume of an MND used in this study is V=1.53×10⁻²¹ m³. The moment of a uniformly magnetized magnetite particle with that volume is:

$$\vert \vec{μ} \vert = V \cdot ρ \cdot {M}_{s} = 1.53 \times {10}^{- 21} {m}^{3} ( \frac{5150 kg}{{m}^{3}} ) ( \frac{85 {Am}^{2}}{kg} ) = 6.7 \times {10}^{- 16} {Am}^{2}$$

Where ρ=5150 kg/m³ and Mₛ=85 Am²/kg are the density and saturation magnetization of bulk magnetite.

Torque:

The torque on a magnetic dipole in a uniform magnetic field is given by³⁸:

[symbol]

= [symbol] × [symbol] ,

where [symbol] is the dipole moment and [symbol] is magnetic field.

Then the torque magnitude is:

| [symbol] |=| [symbol] ∥ [symbol] |

[symbol]

The maximum case occurs when the angle between the magnetic moment and the applied field ( [symbol] ) is 90 degrees, in which case:

| [symbol] |=| [symbol] ∥ [symbol] |=

For magnetic field of 23 mT and a uniformly magnetized MND:

| [symbol] |=6.7×10⁻¹⁶ Am²×0.023 T=1.5×10⁻¹⁷ Nm

The force on the edge of a disk to oppose this magnetic torque scales inversely with the disk radius, so for a 218 nm-diameter disk, that force is:

F=1.4×10⁻¹⁰ N

which is almost three orders of magnitude greater than the force required to activate a mechanosensitive ion channel (˜0.3 pN)³⁹. This analysis provides an upper bound for the force as it assumes that the MND would be completely magnetized in-plane as well as that the MND dipole moment is perpendicular to the field.

A MND 10 degrees off from the applied field would be ˜80% magnetized⁴⁰, and the resulting force would be 20 pN, which is still significantly greater than the response threshold of mechanoreceptors, and thus torque on individual MNDs is a plausible mechanism for magnetomechanical stimulation.

Inter-Particle Attractive Forces

For two magnetic dipoles \vec{μ}₁ and \vec{μ}₂ that are magnetized along the x-axis, and which are also separated by a distance x along the x-axis, the attraction force is:

$$F = \frac{3 {μ}_{0}}{2 π} \frac{\vert {\vec{μ}}_{1} \vert \vert {\vec{μ}}_{2} \vert}{{x}^{4}} \hat{x}$$

If two MNDs are fully magnetized in the x-direction (with their easy axes along the x-direction) and separated by 1 the magnitude of the attraction force is

$$\vert \vec{F} \vert = \frac{3 \times ( 1.26 \times {10}^{- 6} \frac{kg}{{s}^{2}} \frac{m}{{A}^{2}} )}{2 π} \frac{{( 6.7 \times {10}^{- 16} {Am}^{2} )}^{2}}{{( {10}^{- 6} m )}^{4}} = 2.7 \times {10}^{- 13} N$$

This is approximately the force required to activate mechanosensitive ion channels, and is thus also a possible mechanism for the method of magnetomechanical stimulation.

Gradient Force:

The gradient force acting on a magnetic moment in a magnetic field is³⁸:

[symbol]

=∇( [symbol] · [symbol] )

In the case of a magnetic dipole with its moment in the direction of the gradient of [symbol] , the magnitude of the gradient force is:

$$\vert \vec{F} \vert = \vert \vec{μ} \vert \vert \frac{d \vec{B}}{dx} \vert$$

In the case of a MND in the magnetic field simulated in Figure S9, that force is equal to:

| [symbol] |=(6.7×10⁻¹⁶ Am²)×(0.43 T/m)=2.9×10⁻¹⁶N

This is three orders of magnitude lower than the 0.3 pN activation threshold for the mechanoreceptors. Large (˜2 μm diameter) aggregates would be necessary to achieve the forces required to trigger mechanosensitive channels. While such aggregates can in principle occur, our dynamic light scattering spectra of MND solutions, SEM images of DRGs decorated with MNDs, and bright field images of DRGs decorated with MNDs do not indicate prevalence of large clusters. Consequently, gradient force is unlikely to be effective for magnetomechanical modulation in large volumes such as in our experiment.

### Magnetic Energy Calculations

In addition to questioning whether MNDs can produce significant forces in the AMF conditions used in this work, it is also worth considering whether they can transduce an amount of energy comparable to the amount required to trigger a mechanosensitive ion channel. For Piezo1, a known mechanosensor, that energy is ˜40 kT⁴¹.

Zeeman Energy

The potential energy of a magnetic moment in a magnetic field is³⁸:

U=− [symbol] ·

[symbol]

For a MND with its moment aligned with a 23 mT magnetic field, that is:

U=−(6.7×10⁻¹⁶ Am²)(23×10⁻³ T)=−1.5×10⁻¹⁷ J=3.8×10³ kT

This represents the kinetic energy that a MND can produce by rotating from perpendicular to parallel with the applied field, and it is 94× greater than the gating energy of Piezo1.

Shape Anisotropy Energy

A relevant question is whether a MND will physically rotate, or whether its moment will align with the field independent of the particle orientation. To find out, we can consider the shape anisotropy energy, which in this case is the energetic cost of the moment being out of the plane of the MND.

If we approximate a MND as an oblate spheroid with axes a<b=c and c/a=m, we can calculate its demagnetizing factors⁴²:

$${N}_{C} = {N}_{B} = \frac{1}{2 ( {m}^{2} - 1 )} ( \frac{{m}^{2}}{\sqrt{{m}^{2} - 1}} arcsin ( \frac{\sqrt{{m}^{2} - 1}}{m} ) - 1 )$$

$${N}_{A} = 1 - 2 {N}_{C}$$

For a MND of diameter 218 nm and thickness 41 nm, m=5.2.

$${N}_{C} = {N}_{B} = \frac{1}{2 ( {5.2}^{2} - 1 )} ( \frac{{5.2}^{2}}{\sqrt{{5.2}^{2} - 1}} arcsin ( \frac{\sqrt{{5.2}^{2} - 1}}{5.2} ) - 1 ) = 0.12$$

$${N}_{A} = 1 - 2 \times 0.12 = 0.76$$

If we use the equation for the shape anisotropy of a uniaxial particle, we can estimate the energy difference between in-plane and out-of-plane magnetization of a MND (per unit volume):

$$\begin{matrix}Δ {E}_{shape} = \frac{1}{2} {μ}_{0} Δ {NM}_{s}^{2} [ {sin}^{2} {θ}_{1} - {sin}^{2} {θ}_{2} ] \\ = \frac{1}{2} ( 1.26 \times {10}^{- 6} \frac{kg m}{{s}^{2} {A}^{2}} ) ( 0.76 - 0.12 ) {( 438 \times {10}^{3} A / m )}^{2} \\ [ {sin}^{2} ( \frac{π}{2} ) - {sin}^{2} ( 0 ) ] \\ = 7.7 \times {10}^{4} \frac{J}{{m}^{3}}\end{matrix}$$

Per MND, the shape anisotropy energy is:

$${E}_{shape} = ( 7.7 \times {10}^{4} \frac{J}{{m}^{3}} ) ( 1.53 \times {10}^{- 21} {m}^{3} ) = 1.2 \times {10}^{- 16} J = 2.9 \times {10}^{3} kT$$

This energy is an order of magnitude greater than the Zeeman energy, indicating that the moment is trapped in the in-plane orientation and the MND must physically rotate to minimize its Zeeman energy.

Inter-Particle Interaction Energy

The interaction energy of two magnetic dipoles separated by a displacement \hat{r} is³⁸:

$$U = \frac{{μ}_{0}}{4 π} \frac{1}{{r}^{3}} [ {\vec{μ}}_{1} \cdot {\vec{μ}}_{2} - 3 ( {\vec{μ}}_{1} \cdot \hat{r} ) ( {\vec{μ}}_{2} \cdot \hat{r} ) ]$$

In the simple case when the moments are oriented in the 2 direction and also separated along the \hat{x} direction, this reduces to:

$$U = - \frac{{μ}_{0}}{2 π} \frac{1}{{r}^{3}} \vert {\vec{μ}}_{1} \vert \vert {\vec{μ}}_{2} \vert$$

Plugging in for two MNDs one micron apart,

$$U = - \frac{1.26 \times {10}^{- 6} \frac{kg m}{{s}^{2} {A}^{2}}}{2 π} \frac{1}{{( {10}^{- 6} m )}^{3}} {( 6.7 \times {10}^{- 16} {Am}^{2} )}^{2} = 9.0 \times {10}^{- 20} J = 22 kT$$

For two MNDs 500 nm apart,

$$U = - \frac{1.26 \times {10}^{- 6} \frac{kg m}{{s}^{2} {A}^{2}}}{2 π} \frac{1}{{( 5 \times {10}^{- 7} m )}^{3}} {( 6.7 \times {10}^{- 16} {Am}^{2} )}^{2} = 7.2 \times {10}^{- 19} J = 175 kT$$

Thus two MNDs moving from 1 μm to 500 nm apart will produce 4× as much energy as is required for the gating of Piezo1, and inter-particle attraction is a plausible mechanism for magnetomechanical stimulation.

Energy of a MND in a magnetic field gradient

The kinetic energy produced by a MND moving 1 μm down a 0.4 T/m field gradient is 2.9×10⁻²² j=0.07 kT. This means that 565 MNDs moving together would produce enough energy to trigger Piezo1. It is the least energetic MND-field interaction considered and is an unlikely mechanism for magnetomechanical stimulation.

Although the invention has been explained in relation to its preferred embodiment, it is to be understood that many other possible modifications and variations can be made without departing from the spirit and scope of the invention as hereinafter claimed.

### REFERENCES

1. Lee, J.-H. et al. Magnetic nanoparticles for ultrafast mechanical control of inner ear hair cells. ACS Nano 8, 6590-6598 (2014).
2. Huang, H., Delikanli, S., Zeng, H., Ferkey, D. M., and Pralle, A. Remote control of ion channels and neurons through magnetic-field heating of nanoparticles. Nat. Nanotech. 5, 602-606 (2010).
3. Nitsche, M. A. et al. Transcranial direct current stimulation: State of the art 2008. Brain Stimul. 1, 206-223 (2008).
4. Dobson, J. et al., Remote control of cellular behaviour with magnetic nanoparticles. Nat. Nanotechnol. 3, 139-143 (2008).
5. Gupta, A. K. and Gupta, M. Synthesis and surface engineering of iron oxide nanoparticles for biomedical applications. 26, 3995-4021 (2005).
6. Chen, R., Romero, R. Christiansen, M. G., Mohr, A., and Anikeeva, P. Wireless magnetothermal deep brain stimulation. Science 347, 1477-1480 (2015).
7. Li, K., Nejadnik, H., Daldrup-Link, H. E. Next-generation superparamagnetic iron oxide nanoparticles for cancer theranostics. Drug Discov. Today (in press) (2017).
8. Lacroix, L.-M., Carrey, J., and Respaud, M. A frequency-adjustable electromagnet for hyperthermia measurements on magnetic nanoparticles. Rev. Sci. Instrum. 79, 093909 (2008).
9. Feucht, P., Brass, V., and Jordan, A. Alternating magnetic field application device for heating magnetic or magnetizable substances in biological tissue. U.S. Pat. No. 8,688,229 filed 6 Mar. 2009, and issued 1 Apr. 2014.
10. Obeso, J. A. et al. Deep-brain stimulation of the subthalamic nucleus or the pars interna of the globus pallidus in Parkinson's disease. N. Engl. J. Med. 345, 956-963 (2001).
11. Borovikova, L. V. et al. Vagus nerve stimulation attenuates the systemic inflammatory response to endotoxin. Nature 405, 458-462 (2000).
12. Legon, W. et al. Transcranial focused ultrasound modulates the activity of primary somatosensory cortex in humans. Nat. Neurosci. 17, 322-329 (2014).
13. Fenno, L., Yizhar, O., and Deisseroth, K. The development and application of optogenetics. Annu. Rev. Neurosci. 34, 389-412 (2011).
14. Grossman, N. et al. Noninvasive Deep Brain Stimulation via Temporally Interfering Electric Fields. Cell 169, 1029-1041 (2017).
15. Pankhurst, Q. A., Connolly, J., Jones, S. K., and Dobson, J. Applications of magnetic nanoparticles in biomedicine. J. Phys. D: Appl. Phys. 36, R167-R181 (2003).
16. Tay, A., Kunze, A., Murray, C., and Di Carlo, D. Induction of Calcium Influx in Cortical Neural Networks by Nanomagnetic Forces. ACS Nano 10, 2331-2341 (2016).
17. Tay, A. and Di Carlo, D. Magnetic nanoparticle-based mechanical stimulation for restoration of mechano-sensitive ion channel equilibrium in neural networks. Nano Lett. 17, 886-892 (2017).
18. Kim, D.-H. et al. Biofunctionalized magnetic-vortex microdiscs for targeted cancer-cell destruction. Nat. Mater. 9, 165-171 (2010).
19. Shen, Y. et al. Elongated Nanoparticle Aggregates in Cancer Cells for Mechanical Destruction with Low Frequency Rotating Magnetic Field. Theranostics 7,1735-1748 (2017).
20. Cheng, Y. et al. Rotating magnetic field induced oscillation of magnetic particles for in vivo mechanical destruction of malignant glioma. J. Control. Release 223, 75-84 (2016).
21. Banerjee, S. K. and Moskowitz, B. M. in Biomineralization and Magnetoreception in Organisms: A New Magnetism 17-41 (Plenum Press, New York, N.Y., USA, 1985).
22. Howard, J. and Hudspeth, A. J. Compliance of the hair bundle associated with gating of mechanoelectrical transduction channels in the bullfrog's saccular hair cell. Neuron 1, 189-199 (1988).
23. Hughes, S., McBain, S., Dobson, J., and El Haj, A. J. Selective activation of mechanosensitive ion channels using magnetic particles. J. R. Soc. Interface 5, 855-863 (2008).
24. Seo, D. et al. A mechanogenetic toolkit for interrogating cell signaling in space and time. Cell 165, 1507-1518 (2016).
25. Liu, X. L. et al. Magnetic Vortex Nanorings: A New Class of Hyperthermia Agent for Highly Efficient In Vivo Regression of Tumors. Adv. Mater. 27, 1939-1944 (2015).
26. Yang, Y. et al. Orientation mediated enhancement on magnetic hyperthermia of Fe₃O₄ nanodisc. Adv. Funct. Mater. 25, 812-820 (2015).
27. Pichaandi, J., Abel, K. A., Johnson, N. J. J., van Veggel, F. C. J. M. Long-Term Colloidal Stability and Photoluminescence Retention of Lead-Based Quantum Dots in Saline Buffers and Biological Media through Surface Modification. Chem. Mater. 25, 1035-2044 (2013).
28. Brini, M., Cali, T. Ottolini, D., and Carafoli, E. Neuronal calcium signaling: function and dysfunction. Cell. Mol. Life Sci. 71, 2787-2814 (2014).
29. Perea, G. and Araque, A. Glial calcium signaling and neuron-glia communication. Cell Calcium 38, 375-382 (2005).
30. Aravanis, A. M. et al. An optical neural interface: in vivo control of rodent motor cortex with integrated fiberoptic and optogenetic technology. J. Neural Eng. 4, S143-S156 (2007).
31. Delmas, P., Hao, Jizhe., and Rodat-Despoix, L. Molecular mechanisms of mechanotransduction in mammalian sensory neurons. Nat. Rev. Neurosci. 12, 139-153 (2011).
32. Bouron, A., Kiselyov, K., and Oberwinkler, J. Permeation, regulation and control of expression of TRP channels by trace metal ions. Pflugers Arch., EJP 467, 1143-1164 (2015).
33. Cahusac, P. M. B. Effects of transient receptor potential (TRP) channel agonists and antagonists on slowly adapting type II mechanoreceptors in the rat sinus hair follicle. J. Peripher. Nerv. Syst. 14, 300-309 (2009).
34. Coste, B. et al., Piezo1 and Piezo2 are essential components of distinct mechanically activated cation channels. Science 330, 55-60 (2010).
35. Hanani, M. Role of satellite glial cells in gastrointestinal pain. Front. Cell. Neurosci. 9, 412 (2015).
36. Kim, D. et al. Synthesis of uniform ferromagnetic magnetite nanocubes. J. Am. Chem. Soc. 131, 454-455 (2009).
37. Jia, H. Rochefort, N. L., Chen, X., and Konnerth, A. In vivo two-photon imaging of sensory-evoked dendritic calcium signals in cortical neurons. Nat. Protoc. 6, 28-35 (2011).
38. Griffiths, D. J. Magnetic Fields in Matter. In Introduction to Electrodynamics Edn. 3 (eds. Reeves, A. & Dellas, K.) 257-282 (Prentice Hall, Upper Saddle River, N.J., USA, 1999).
39. Howard, J. and Hudspeth, A. J. Compliance of the hair bundle associated with gating of mechanoelectrical transduction channels in the bullfrog's saccular hair cell. Neuron 1, 189-199 (1988).
40. Yang, Y. et al. Orientation mediated enhancement on magnetic hyperthermia of Fe₃O₄ nanodisc. Adv. Funct. Mater. 25, 812-820 (2015).
41. Cox, C. et al. Removal of the mechanoprotective influence of the cytoskeleton reveals PIEZO1 is gated by bilayer tension. Nat. Commun. 7, (2016).
42. Cullity, B. D. and Graham, C. D. Experimental Methods. In Introduction to Magnetic Materials. Edn. 2 (Hanzo, L. et al.) 52-55 (John Wiley & Sons, Inc., Hoboken, N.J., USA 2009).

While this invention has been particularly shown and described with references to preferred embodiments thereof, it will be understood by those skilled in the art that various changes in form and details may be made therein without departing from the scope of the invention encompassed by the appended claims.

1. A method for mediating remote control of cell signaling in a biological sample, comprising the steps of:
(i) contacting the biological sample with magnetic nanodiscs; and
(ii) applying a magnetic field to the biological sample,
wherein the applied magnetic field is of low amplitude and low frequency.

2. The method of claim 1, wherein the biological sample is selected from a population of cells, an organ, an organ explant, a tissue, and a tissue explant.

3. The method of claim 2, wherein the sample comprises dorsal root ganglion explants.

4. The method of claim 3, wherein the sample comprises neuron and glial cells.

5. The method of claim 4, wherein the neuron cells are selected from Purkinje cells, Granule cells, Motor neurons, Tripolar neurons, Pyramidal cells, Chandelier cells, Spindle neurons, and Stellate cells.

6. The method of claim 1, wherein the magnetic nanodiscs mediate cell signaling via a mechanosensitive channel or a mechanosensitive ion channel.

7. The method of claim 6, wherein the mechanosensitive channel or the mechanosensitive ion channel is selected from an anion and a cation channel.

8. The method of claim 7, wherein the channel is a calcium, sodium, potassium, or chloride channel.

9. The method of claim 1, wherein the magnetic nanodiscs are biocompatible.

10. The method of claim 1, wherein the magnetic nanodiscs are anisotropic.

11. The method of claim 1, wherein the magnetic nanodiscs are comprised of magnetite.

12. The method of claim 1, wherein the magnetic nanodiscs exhibit colloidal stability.

13. The method of claim 1, wherein the magnetic field is weak.

14. The method of claim 1, wherein the frequency of the magnetic field is slow-varying.

15. The method of claim 1, wherein the amplitude is between 5 and 50 millitesla.

16. The method of claim 1, wherein the frequency is between 1 and 10 hertz.

17. The method of claim 1, wherein the amplitude is 23 millitesla, and the frequency is 5 hertz.

18. The method of claim 1, wherein the nanodiscs have a concentration of magnetic nanomaterials of between 3 μg/mL and 300 μg/mL.

19. The method of claim 1, wherein the nanodiscs have a concentration of magnetic nanomaterials less than 100 μg/mL.

20. The method of claim 1, wherein the method is scalable to large volumes without deleterious effects on the biological sample.

21. The method of claim 20, wherein the volume is greater than 500 cm³.

22. The method of claim 1, wherein the diameter of the magnetic nanodiscs is less than 300 nm.

23. A device for mediating remote control of cell signaling in a biological sample in the presence of a weak magnetic field of low amplitude and slow-varying frequency.

24. The device of claim 23, wherein said device is scalable.

25. The device of claim 23, wherein said device provides for remote transduction of magnetomechanical stimuli in a cell membrane of the sample.

26. The device of claim 25, wherein said membrane comprises a mechanosensitive channel or a mechanosensitive ion channel.

27. The device of claim 23, wherein the device is a magnetic nanodisc.

28. The device of claim 26, wherein the mechanosensitive channel or a mechanosensitive ion channel is selected from an anion and cation channel.

29. The device of claim 28, wherein the channel is a calcium, sodium, potassium, or chloride channel.

30. The device of claim 23, wherein the device is biocompatible.

31. The device of claim 23, wherein the device is anisotropic.

32. The device of claim 23, wherein the device is comprised of magnetite.

33. The device of claim 23, wherein the device exhibits colloidal stability.

34. The device of claim 23, wherein the amplitude is between 5 and 50 millitesla, and the frequency is between 1 and 10 hertz.

35. The device of claim 34, wherein the amplitude is 23 millitesla, and the frequency is 5 hertz.

36. The device of claim 23, wherein the nanodiscs have a concentration of magnetic nanomaterials of between 3 μg/mL and 300 μg/mL.

37. The device of claim 23, wherein the nanodiscs have a concentration of magnetic nanomaterials less than 100 μg/mL.

38. The device of claim 23, wherein the diameter of the magnetic nanodiscs is less than 300 nm.

39. A method for remote transduction of magnetomechanical stimuli in a cell membrane of a biological sample, comprising the steps of:
(i) contacting the biological sample with magnetic nanodiscs; and
(ii) applying a magnetic field to the biological sample,
wherein the applied magnetic field is of low amplitude and low frequency.

40. The method of claim 39, wherein the sample is selected from a population of cells, an organ, an organ explant, a tissue, and a tissue explant.

41. The method of claim 40, wherein the sample comprises dorsal root ganglion explants.

42. The method of claim 41, wherein the sample comprises neuron and glial cells.

43. The method of claim 42, wherein the neuron cells are selected from Purkinje cells, Granule cells, Motor neurons, Tripolar neurons, Pyramidal cells, Chandelier cells, Spindle neurons, and Stellate cells.

44. The method of claim 39, wherein the magnetic nanodiscs mediate cell signaling via a mechanosensitive channel or a mechanosensitive ion channel.

45. The method of claim 44, wherein the mechanosensitive channel or the mechanosensitive ion channel is selected from an anion and a cation channel.

46. The method of claim 45, wherein the channel is a calcium, sodium, potassium, or chloride channel.

47. The method of claim 39, wherein the magnetic nanodiscs are biocompatible.

48. The method of claim 39, wherein the magnetic nanodiscs are anisotropic.

49. The method of claim 39, wherein the magnetic nanodiscs are comprised of magnetite.

50. The method of claim 39, wherein the magnetic nanodiscs exhibit colloidal stability.

51. The method of claim 39, wherein the magnetic field is weak.

52. The method of claim 39, wherein the frequency of the magnetic field is slow-varying.

53. The method of claim 39, wherein the amplitude is between 5 and 50 millitesla.

54. The method of claim 39, wherein the frequency is between 1 and 10 hertz.

55. The method of claim 39, wherein the amplitude is 23 millitesla, and the frequency is 5 hertz.

56. The method of claim 39, wherein the nanodiscs have a concentration of magnetic nanomaterials of between 3 μg/mL and 300 μg/mL.

57. The method of claim 39, wherein the nanodiscs have a concentration of magnetic nanomaterials less than 100 μg/mL.

58. The method of claim 39, wherein the method is scalable to large volumes without deleterious effects on the biological sample.

59. The method of claim 58, wherein the volume is greater than 500 cm³.

60. The method of claim 39, wherein the diameter of the magnetic nanodiscs is less than 300 nm.

61. A method to treat cancer in a biological sample or subject, comprising the steps of:
(i) contacting the cancer with magnetic nanodiscs; and
(ii) applying an alternating magnetic field to the cancer, and
wherein the applied magnetic field is of low amplitude and low frequency.

62. The method of claim 61, wherein the method is scalable to large volumes.

63. The method of claim 62, wherein the volume is greater than 500 cm³.

64. The method of claim 61, wherein the amplitude of the applied magnetic field is between 5 and 50 millitesla, between 10 and 40 millitesla, or between 20 and 30 millitesla.

65. The method of claim 61, wherein the frequency is between 1 and 10 hertz, or between 3 and 7 hertz.

66. The method of claim 61, wherein the amplitude of the applied magnetic field is 23 millitesla, and the frequency is 5 hertz.

67. The method of claim 61, wherein the magnetic nanodiscs are biocompatible, anisotropic, and comprised of magnetite.
